(19) United States
(12) Patent Application Publication
(10) Pub. No.: US 2017/0023555 A1
(43) Pub. Date: Jan. 26, 2017

(54) DEVICES AND KITS FOR MEASURING BIOLOGICAL RESULTS

(71) Applicant: Credo Biomedical Pte Ltd., Singapore (SG)

(72) Inventors: Chung-Pei OU, Singapore (SG); Abdur Rub Abdur RAHMAN, Singapore (SG); Kaushal SAGAR, Singapore (SG); Stephen Chang-Chi KAO, Singapore (SG); Winston WONG, JR., Singapore (SG)

(21) Appl. No.: 15/302,493

(22) PCT Filed: Apr. 10, 2015
(86) PCT No.: PCT/IB2015/001424

§ 371 Date: Oct. 7, 2016

Related U.S. Application Data
(60) Provisional application No. 61/978,486, filed on Apr. 11, 2014.

(51) Int. Cl. G01N 33/52; G01N 33/84; C12Q 1/68

(52) U.S. Cl. CPC G01N 33/52; C12Q 1/6813; G01N 33/84

(57) ABSTRACT

The present invention relates to a kit and a device for measuring nucleic acid amplification through colour differentiation wherein said kit contains at least one pH indicator dye, one or more contained amplification reagents. The kit and device of the present invention also are used to detect, measure and/or record enzymatic reactions that result in pH changes. The kit and device provide a mechanism to detect pH change by utilizing a pH indicator dye, thus making it observable with the un-aided eye. The kit contains a device for carrying out said reactions. The device contains at least one container, reagents, a pH indicator, a heating or cooling means where needed and a magnetic component.

### BACKGROUND OF THE INVENTION

The present invention relates to kits and devices for diagnostic, genetic testing, pedigree and breed selection testing, genetic modified organism testing, pathogen detection, genotyping, mutation detection, companion gene testing for prescription or clinical treatment, detection of cancer type, monitoring and prognosis of cancer via the use of nucleic acids and enzymatic and other biological and chemical reactions that result in pH changes. In particular, pH changes such as pH meters are used to detect, measure and/or record many chemical and/or biological reactions. The present invention relates to devices and kits wherein these reactions are carried out and easily and efficiently measured.

Nucleic acid analysis has been widely used in clinical application, the food industry, forensic testing, human identification, pathogen epidemic surveillance and detection of new disease strains. This genetic testing covers a range of technologies that involve detection and identification of nucleic acids from analytes. Examples includes DNA sequencing, real-time polymerase chain reaction (PCR), DNA microarray, and restriction fragment length polymorphism (RFLP), as examples. The present invention provides enhanced means via a kit and specified devices by which to carry out such testing.

Traditional methods for detecting nucleic acids that are often times found in minute quantities require multiple devices and steps to process a sample, amplify the target, and detect the amplification. Amplification of nucleic acids, DNA or RNA, has been well established, and there are various methods that exist today for different assay requirements. Thermocycling based Polymerase-Chain-Reaction (PCR) based amplification has been shown to be reliable in detecting nucleic acids, as well as gene variations, such as copy number variation or single-nucleotide polymorphism. This method has been well established such that it is often times a standard method for applications that require most regulation such as clinical and forensic applications. Regardless of the nucleic acid amplification methods, the amplified products are not detectable without a visualization method. Current nucleic acid visualization methods and kits relate to attaching a fluorescent probe to the amplification reaction. These probes include the fluorescence tag in a Tagman detection oligo and double-stranded DNA chelator, Sybr Green or other fluorescence chemical that is sensitive to the reaction product. The fluorescence compound is essential in this type of detection because of the high proton emission from the fluorescent molecule, and the emission is only detectable in the presence of the reaction product. The emission only occurs when the fluorescence probe of the Tagman detection oligonucleotide is hybridized to the amplified product and cleaved by the DNA polymerase, or the Sybr Green is chelated to the amplified product.

However, these fluorescent chemicals are sensitive to light exposure or require special storage conditions such as refrigeration. Exposure to the ambient light causes irreversible damage to the fluorescence chemicals, a phenomenon called photo bleaching. For any fluorescence method, an excitation light source would also be required for any emission to occur. An UV light source is normally used as the excitation light source, to excite the fluorescence probe in order to produce measurable light emission. One example is published by Paul LaBarre of PATH, Seattle, USA (PloS One V6, issue 6, e19738), incorporated in its entirety by reference. Fluorescence emission is possible when the amplification product, pyrophosphate, relieves the fluorescence chemical from being quenching. An UV light source is needed and is provided by a handheld UV LED. The intensity of the light depends on the quantity of the product and the ambient light condition. In the case of comparing an unknown sample to a positive control and a negative control, the single UV LED would not be able to provide uniform illumination to all three samples. It could be hard to differentiate the positive response from a negative one without the help from an instrument. Often inconsistent emission from the fluorescence dye occurs. As the emission relies on the swap between two metal ions binding, which is a secondary reaction other than the amplification reaction, it is subject to interference from other metal chelators commonly existing in blood mixed with EDTA to prevent clotting or other operation variations.

Another example where the sample could inhibit or prevent the fluorescence reading is when the solution is not a clear solution. In one particular example, the sample is untreated whole blood. When 2 micro litre of the blood is mixed with 50 micro litre of the reaction, the reaction mix is cloudy. Without precision instruments, it is nearly impossible to handle the sample volume less than 1 micro litre. While most of the nucleic acid reaction is performed under 50 micro litre, more commonly at 25 or 10 micro litre, when the sample is cloudy or strongly coloured, the fluorescence methods are severely restricted. Large dilution or a purification step is required prior to the reaction.

An amplification method for detecting nucleic acids using a pH sensitive system directly measures hydrogen ions rather than using fluorescent dyes. This is accomplished by utilizing CMOS chip technology with an ion-sensitive effect transistor (ISFET) sensor Toumazou, Christofer, et al., Nature Method, 2013, Vio(7) p 641. The hydrogen ion sensing layer is the silicon nitride which is the top layer of a CMOS chip. This technology results in cost-effective, nucleic acid analysis. It is essential to be electrically connected for any CMOS chip, and special packaging of the chip is needed to allow the measurement and amplification reaction. As a consequence, the method is expensive and challenging. It is expensive because of the high cost associated with both the design and production of any CMOS chip. It is challenging because of at least two reasons: 1. the risk of short circuit from the amplification liquid leakage via pin hole or minor packaging defect, and 2. The risk of strong interference between the sensing layer, e.g. silicon nitride, and the reaction components. Because of these challenges and concerns, there is a need for a genetic test kit that is cost effective and simple, and new devices for use in such kits are needed.

### SUMMARY OF THE INVENTION

The present invention provides such kits and devices for biological and/or chemical reactions preferably for nucleic acid detection, protein detection and other chemical and/or biological detection and/or measurement means, as well as enzymatic reactions that result in pH changes.

The identification of nucleic acid sequences or nucleotides is typically achieved by using sequencing techniques to provide the sequence data. However, the genetic analysis using sequencing method of the art are laborious because the sequencing process is a systematic effort to provide ultra-pure nucleic acid, precision detection via enzymatic or physical methods, and powerful computation for decoding the vast information produced during the detection step. When polymerase enzyme is used in the detection, one or more nucleotides are incorporated by the polymerase. The identity of the sequence is decoded by the order of the nucleotides added to the detection reaction. Nucleotide sequences are also determined by sequence-specific detection methods, such as hybridization and/or nucleic acid amplification. These techniques typically involve the use of one or more short oligonucleotides of known nucleotide sequences. The use of the oligonucleotides greatly reduces the complexity of generating any genetic analytical steps. In these situations, a desktop device is sufficient to provide the nucleic acid sequence analysis. Also, automatic devices provide the tested genetic result directly from a sample. Typically, the oligonucleotides of the sequence-specific detection method found in the art corresponds to a small fraction of the sample genetic makeup. Oftentimes, enzymes, such as polymerases, or physical methods, such as the fluorescence method discussed hereinabove are cited.

In yet another example of analysis for nucleic acids, the process is mediated by restriction enzyme such as the Invader Assay from Beckman Coulter. When a polymerase is involved in the testing, one or more nucleotides are incorporated to the oligonucleotides of the test kit. Such a method is found in the primer extension assay used in the Infinium assay from Illumina. Another example is a Taqman assay that utilizes a polymerase chain reaction (PCR) to detect a single nucleotide polymorphism (SNP) as discussed previously. Furthermore, recent developments in isothermal amplification, e.g. loop-mediated amplification (LAMP) and recombinase polymerase amplification (RPA) have simplified the amplification process without the need for the precision thermal cycling step.

There are many examples of an enzyme activity used in nucleotide identification. Such examples are restriction cutting in an Invader® assay, strand displacement in the use of LAMP, recombinase in RPA, and polymerization in PCR. The product of these enzymatic actions typically is detectable by incorporating fluorescent labeled nucleotides or fluorescence dyes that are sensitive to the enzymatic product. An additional light source is provided at a shorter wavelength to excite each fluorescent component, which, upon excitation, emits light at a longer wavelength. Upon excitation, the intensity of the emitted light typically increases proportionally to the enzymatic product. However, these techniques are deficient in that the intensity from one run of the assay is not repeated when another run of the assay is used. It is thus challenging to differentiate a positive reaction from a negative one in these assays. One way to overcome this is by including a panel of sample controls and by adapting an optical sensor to measure light emission. In these situations, a sensor is typically fitted with a light filter to prevent interference from the excitation light source. In the case of a fluorescent compound used in such LAMP situations, the excitation light source is an ultraviolet lamp that may be harmful to the user and thus, a light shield must provided to prevent the effects of harmful radiation.

The present invention thus relates to a kit that avoids the optical and measurement challenges associated with fluorescent components used in existing assays and kits. The kits of the invention include devices for carrying out the methods of the invention.

The kit of the present invention comprises at least one pH indicator dye that is typically immobilized on a solid surface, such as a bead or film. It is known that enzymatic reactions produce changes in proton concentration. The proton concentration changes during hydrolysis of nucleotides, incorporation of the nucleotides to the oligonucleotides, polymerization of the nucleotides, and hydrolysis of the ether bond, are just a few examples. The colour of the pH indictor changes upon the change of the proton concentration. Typical pH indicators exist as several chemical species with varied protonation in any point in time. Each chemical species of the pH indicator has a distinct number of protonated sites. Each chemical species thus has a distinct optical spectrum. When there is more than one isosbestic point or equivalent in the spectra superposition of the species, the indicator dye has different colours at different pH value. The spectrum is typically made up of a few peaks at different wavelength. For observing the spectrum change, a minute intensity change at any peak alone cannot be detected by the unaided eyes. On the other hand, a combination of a minute decrease on one spectrum peak and a minute increase on another produces a new colour which is easily detected by the unaided eyes. One example of the colour-changing pH indicator dye is potassium 4-[4-(2-hydroxyethanesulfonyl)-phenylazo]-2, 6-dimethoxyphenol (K2 dye) which is yellow when fully protonated. The K2 dye changes to magenta when fully de-protonated. Its colour is orange when half the protonation sites are protonated.

The opposite examples are pH indicator dyes that do not have isosbestic points on the spectra versus a pH graph, such as p-nitrophenol or fluorescein. In the case of p-nitrophenol, the colour is a strong yellow, pale yellow or colourless depending on the pH value. It is very challenging to recognize the yellow grade by the unaided eyes, if pH change is 1 or less.

The present kit accomplishes these results and comprises a kit with at least one pH indicator dye that changes upon pH change. Preferably, in the kit, the dye component is pre-loaded in the reaction chamber, such as a PCR tube, an 8-tube PCR strip, a 96-well plate, or provided in a dispenser but may be on beads. When there is an amplification reaction after adding the sample, the pH change causes the colour change of the pH indicator dye. The colour change is much easier to see by the un-aided eye when the dye is immobilized to a solid matrix, where it is permeable to the hydrogen ion but not DNA polymerase or nucleic acids. Because of differential permeability, it is possible to increase the optical density by increasing the dye concentration in the solid matrix without increasing the risk of inhibiting the reaction. The pH indicator could be particles or immobilized to the particles. The size of the particles is not limited by the selection of the dye or colour. The size of the particles is only relevant to the choice of the reaction container or condition. The dye could also be immobilized on a film or the surface of the container such that it minimizes the interference to the amplification reaction.

The present kit invention has a pH sensitive dye used to detect or monitor nucleic acid amplification. Use of beads may be advantageously used in the kits of the present invention. The beads are spherical particles synthesized from any suitable material for the attachment of the dye, e.g. silica, polystyrene, agarose or dexteran. The particles can be synthesized using a core-shell structure, and as such, a particle can be formed by both paramagnetic materials and a dye hydrogel. The bead from silica, for example, has higher density such that it is easy to keep the bead at a constant position in the solution or moving the bead out of the solution by inverting the reaction vial.

Furthermore, the present invention relates to and provides devices and/or machines that are designed to run the reaction of the invention and measurements to record the reactions thereof.

The present invention therefore provides a device that detects pH changes as indicated herein, with a heating system, if needed, for the reaction to be carried out by the device. This device is then able to detect pH changes in real time.

As one object of the present invention, colorimetric detection of pH based changes, in real time, and/or nucleic acid amplification reactions are provided, (see US Patent Application No. PCT/IB2014/002637 incorporates herein by reference in its entirety wherein such reactions are described) and U.S. Provisional patent applications 61/873,463 and 61/919,881 also incorporated herein by reference in their entirety.

Another object of the present invention relates to the use of pH dye-based detection to measure pH-based amplification and/or colorimetric detection of pH changes in real time. See U.S. patent Ser. No. 13/618,694 incorporated herein by reference in its entirety and U.S. Provisional Patent Application 61/535,874 incorporated herein by reference in its entirety.

These objects of the present invention are accomplished on a device that may additionally have a magnet present in a location that is in the same reaction container or in a different location than the reaction container, such as in a different reaction t vessel, vessel, container, chamber, reaction chamber vial, vessel, test tube or tube. (All considered interchangeable herein.)

As indicated above, the present invention also includes a device in which a sample preparation and the resultant reaction are carried out in the same vessel, container, chamber, vial and/or tube. The container (vessel, vial or tube) in which the reaction is carried out can already have magnetic beads in place. This avoids any contamination issues and also avoids using a magnet in another container.

The device of the present invention also can include a computer with artificial intelligence. Such a device/computer combination is useful for many functions. One such function is the ability to measure and/or determine when the reaction being run has come to an end or has been completed. Another use of the combined device with artificial intelligence computer is to take measurements of a positive control of the reaction being measured, negative control of the reaction being measured an/or both positive and negative controls. Additionally, such a combination device can be used for real time observations and providing instructions on how and when to proceed.

Artificial intelligence can take advantage of mathematical manipulations of the time series of signals from the same container or across different containers as inputs to the built in algorithm. Examples include but are not limited to taking derivatives and integral of time series and taking differential manipulation of time series from one or more containers from the same or different time points.

Oftentimes, a camera is connected to the artificial intelligence means in order to visualize the progress of the biological reaction and provide input for controlling the reaction.

These and further objects of the invention will become known with the detailed description of the invention and description of the figures provided hereinbelow.

### BRIEF DESCRIPTION OF THE FIGURES

FIG. 1. The colour of each dye film corresponds to a pH range.

FIG. 2. The photo illustrated the colour response of the pH film in a LAMP reaction for 2C19 genotyping.

FIG. 3. K1 chemical is tested in the form of a film, cellulose particles and soluble molecules.

FIG. 4. The photo shows the colour of the dye in each tube prior to the LAMP reaction.

FIG. 5. This photo shows the colour of the dye change in the tube where amplification occurs in the LAMP reaction in the top row while the colour of the dye is unchanged where there is not amplification in the LAMP reaction in the bottom row.

FIG. 6. This shows two distinct films for amplification detection testing.

FIG. 7. Bromothysial blue dose not produce a colour change and the pH remains unchanged.

FIG. 8. The dye colour of each tube is pink prior to the LAMP reaction.

FIG. 9. The dye colour then changes to yellow for tubes 1 to 7 and remains pink for tubes 8-10.

FIG. 10. This chart shows the positive and negative discrimination response.

FIG. 11. These are agarose electrophoresis photos showing the LAMP amplification in lanes 1 to 7.

FIG. 12. This shows the dye colour prior to the reactions that are positive or negative with regard to DNA.

FIG. 13. This shows the dye colour after to the reactions that are positive or negative with regard to DNA.

FIG. 14. This is a whole blood effect on dye colour prior to the reaction.

FIG. 15. This is a whole blood effect after the reaction.

FIG. 16. This shows the colour of the immobilized dye after shaking the solution off the dye.

FIG. 17. This is s LAMP reaction from each tube using agarose electrophoresis.

FIG. 18. This shows the result of a PCR reaction with the presence of dye.

FIG. 19. This is a schematic of the physical entrapment and chemical linkage pH indicator dye to the cross-linked polymer matrix.

FIG. 20. This shows the colour difference between the reaction versus no reaction when hydrogel slabs are used.

FIG. 21. This shows an example of a pH responsive dye conjugated hydrogel of polyurethane on a cellulose acetate ball of 2 mm diameter.

FIG. 22. Lamp figure result.

FIG. 23. Service for housing kit components and device of the present invention.

FIG. 24. Device with heating medium such as wax.

FIG. 25. Device with different heating units.

FIG. 26. Device with reading guide.

FIG. 27. Device with reading guide and lens.

FIG. 28. Device with readout potentials.

FIG. 29. Device with light source and readout embodiment.

FIG. 30. Device with colour sensor.

FIG. 31. Device with reaction chamber for dye positioning.

FIG. 32. Device with dye on surface of bead.

FIG. 33. Device with use of large beads.

FIG. 34. Device with use of small beads.

FIG. 35. Device with metal or magnetic beads.

FIG. 36. Device with use of paramagnetic or ferromagnetic compounds.

FIG. 37. Device with magnet to separate beads or keep them in place.

FIG. 38. Device to control bead locations.

FIG. 39. Device with further magnet or metal box to control bead locations.

FIG. 40. Movable device component for magnet or metal box transport.

FIG. 41. Magnet or metal box location after reaction is completed.

FIG. 42. Further location for magnet or metal loop after reaction is complete.

FIG. 43. Device with active thermal and, dye solution container, magnet and light sources.

FIG. 44. Provides results of the colorimetric SNP reaction when the sample is saliva.

FIG. 45. This provides the results of the present invention with the use of LAMP with fluorescence.

FIG. 46. The primers used in the reactions in FIGS. 44 and 45 are provided here.

FIG. 47. The reagents for the reaction are provided for the colorimetric display.

FIG. 48. The reagents for the florescence reaction are provided in this chart.

FIG. 49: This figure provides the example parameters for testing a whole blood sample.

FIG. 50: This provides the results when a whole blood sample containing genotype 2G is measured by the device of the present invention.

FIG. 51: This figure provides an example of the device of the invention.

### DETAILED DESCRIPTION OF THE INVENTION

Detection by pH change in nucleotide identification has been shown by using sequencing enzymatic synthesis, (Rothberg et al 20111, Nature, 475, p 348) and LAMP (Toumazou et al 2013, Nature Methods, 10, p 641) incorporated herein by reference. In these methods, one or more electrical sensors are used to detect the pH value of the nucleotide identification reactions. These sensors have a very small surface area, typically in the micrometer range. The miniature sensor development makes it viable to monitor the nucleotide identification reaction without completely inhibiting the enzyme activity. However, it is technically very challenging to create a physical barrier to isolate each sensor from the others. It only becomes viable when there is an economy of scale for the manufacturing of the barriers.

As described herein, the present invention provides devices and kits to detect one or more targets, including biological, chemical, or material targets. In part, this is accomplished through the use of stable and robust enzymatic systems that allow direct detection of a biological target and/or changes in pH. Detection time is much reduced, with sample-to-result times of less than 1 hour or as short as 15 minutes. The enzymes used in the present invention are preferably stable at room temperature. In some embodiments, the invention enables detection without sophisticated instrumentation, thus making the invention amenable to point of care (POC) applications.

Accordingly, the devices and kits of the present invention provide for significantly reduced setup costs and equipment requirements for point of care detection and are amenable for application to a disposable kit.

It is to be understood that the present invention is not limited to particular methods, reagents, compounds, compositions or biological systems, which can, of course, vary. It is also to be understood that the terminology used herein is for the purpose of describing particular aspects only, and is not intended to be limiting. As used in this specification and the appended claims, the singular forms “a”, “an” and “the” include plural references unless the content clearly dictates otherwise.

The term “about” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein.

As is well known the art, the chromatographic medium may be cast onto the support material wherein the resulting laminate may be die-cut to the desired size and shape. Alternatively, the chromatographic medium may simply be laminated to the support material with, for example, an adhesive. In some embodiments, a nitrocellulose or nylon porous membrane is adhered to a film.

An “indicator” refers to any of various substances, such as litmus, phenolphthalein, or bromothymol blue, Potassium I-hydroxy-4-[1-(2-hydroxyethylsulphonyl) phenylazo]-naphthalene-2-sulphonate, cellulose acetate coupled potassium I-hydroxy-4-[1-(2-hydroxyethylsulphonyl) phenylazo]-naphthalene-2-sulphonate and the like that indicate the presence, absence, or concentration of another substance or the degree of reaction between two or more substances by means of a characteristic change, especially in color.

A “sample” refers to any source which is suspected of containing an analyte or target molecule. Examples of samples which may be tested using the present invention include, but are not limited to, blood, serum, plasma, urine, saliva, cerebrospinal fluid, lymph fluids, tissue and tissue and cell extracts, cell culture supernatants, biopsy specimens, paraffin embedded tissue, soil, fruit, juice, oil, milk, food, water, among others. A sample can be suspended or dissolved in liquid materials such as buffers, extractants, solvents, and the like.

“Proficient enzyme” or “high yield enzyme” refers to an enzyme that can generate a product at a high rate that approaches the diffusion limit.

A “proficient enzyme conjugate” refers generally to a proficient enzyme, which is conjugated to a reporting carrier. The nature of the interaction is covalent or non-covalent or a hybrid of both.

The kit of the present invention therefore preferably includes the pH detector in the solution during the chemical reaction and can be in solution, in a cocktail of reagents and/or lyophilized either alone or with other reagents. It is further preferable that a physical barrier to separate one reaction from another in order to prevent contaminating the reading result be part of the detection device of the kit and device. Also, beads containing the dye on them, beads in the dye solution or magnetic beads with the dye on them are used in the present kit and device.

In the present kit, the use of an immobilized pH indicator(s) on enzyme-friendly surfaces is found. These pH indicators can be cross linked to various materials such as cellulose acetate or hydrogel. The indicators have a pKa value that is between 6 and 9, and as such are useful to indicate the absolute pH value in a reaction. It is not practical to perform the nucleotide identification by use of the kits of the present invention by using soluble pH indicators, because of the use of the pH indicators having many drawbacks. The colour intensity follow the Beer-Lambert law which is proportional to the concentration of the indicator and the light path. A typical pH indicator, such as Creso red, bromothymol blue, or phenol red would inhibit a LAMP reaction at a typical working concentration (0.2-1 mg/mL). Lowering the concentration will reduce the colour intensity which follows the Beer-Lambert law. In a miniature device or reaction chamber, it is very challenging to recognize the colour by the unaided eye when the concentration of the dye is so low.

When working with an immobilized pH indicator, the colour intensity depends on the dye density on the surface of the material, which typically requires no more than 10-20 micro meter in depth, which is well accessible to the proton in the solution. The immobilization allows intense colour without inhibiting the enzyme activity. The immobilized pH indicator can be made compatible to the PCR, where if dye has any inhibitory interference, the inhibition will be accelerated and aggravated due to the high temperature and rapid mass transport rate.

The colour intensity can be further increased by rendering the spectroscopy properties of the surface. The opacity or intrinsic colour can be adjusted such that a better colour contrast could be obtained after immobilization of pH indicator. For example, the K2 is light yellow when fully protonated. The yellow is vivid visible when it is conjugated to a white opaque surface. It also makes machine colour reading easier and more accurate.

We have shown it is possible to immobilize a pH indicators on cellulose acetate surface or hydrogel as a component of the kit and device in the kit. The materials where a pH indicator could be immobilized to are not limited by the described materials.

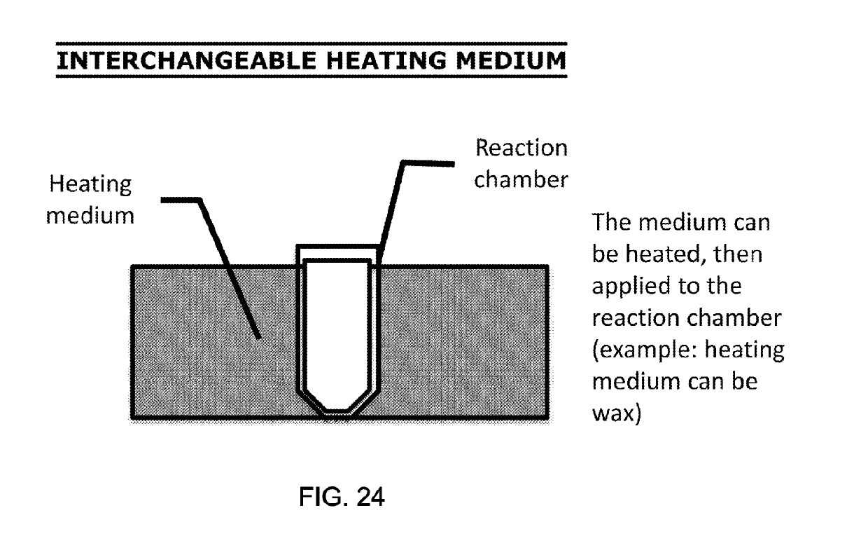

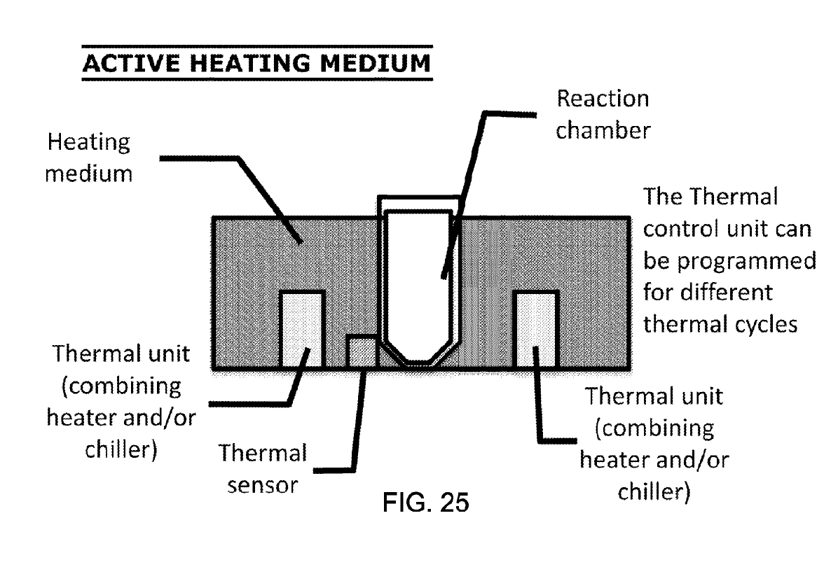

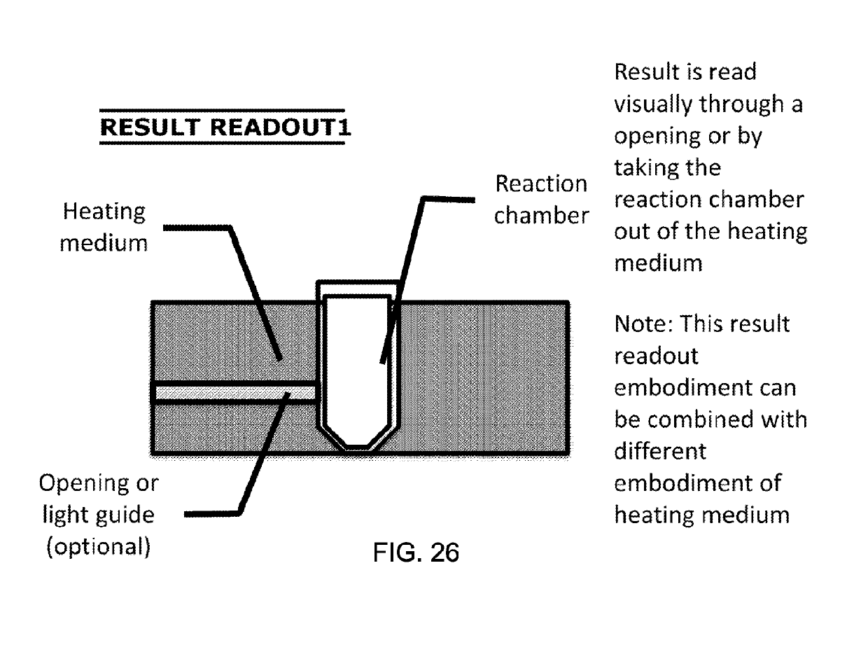

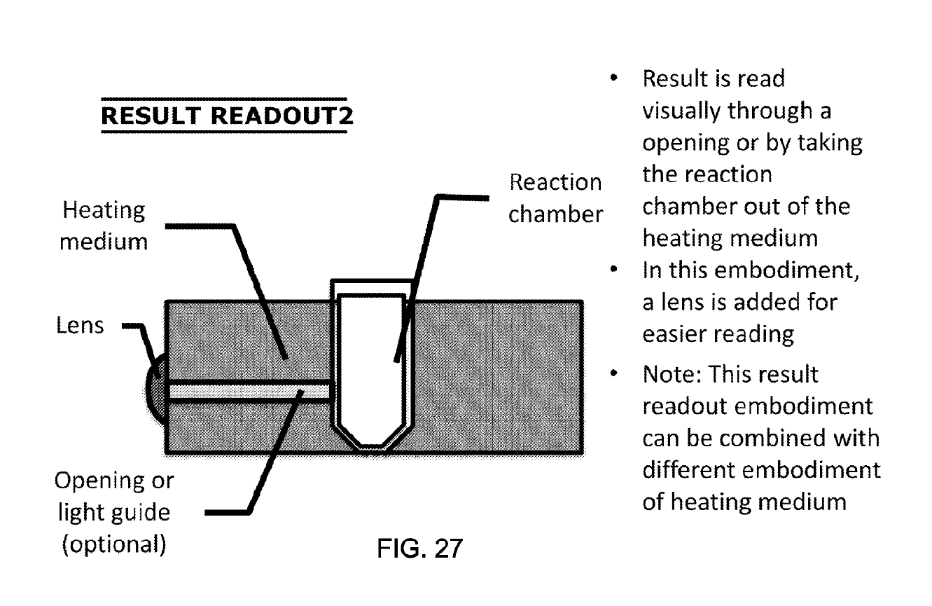

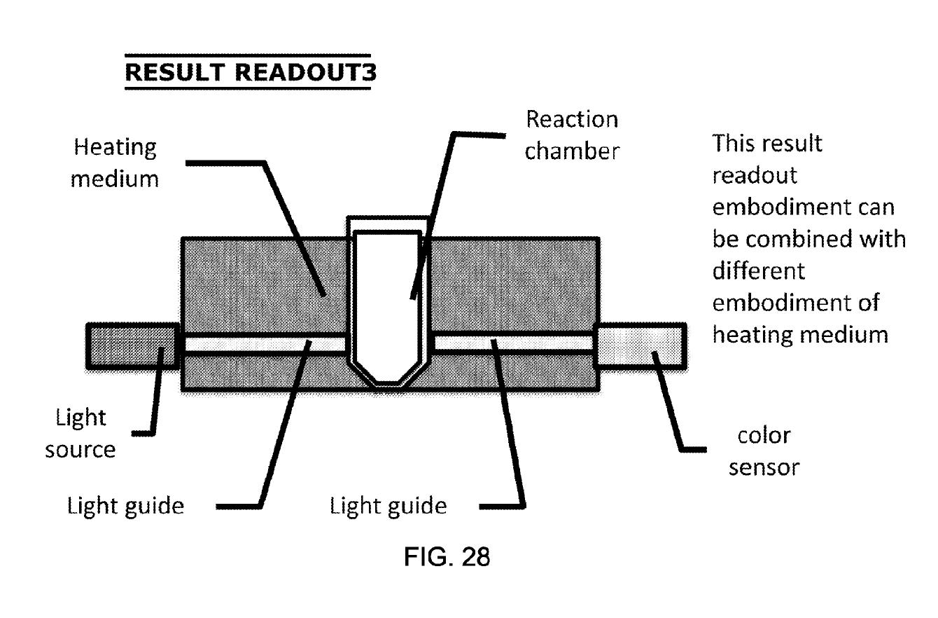

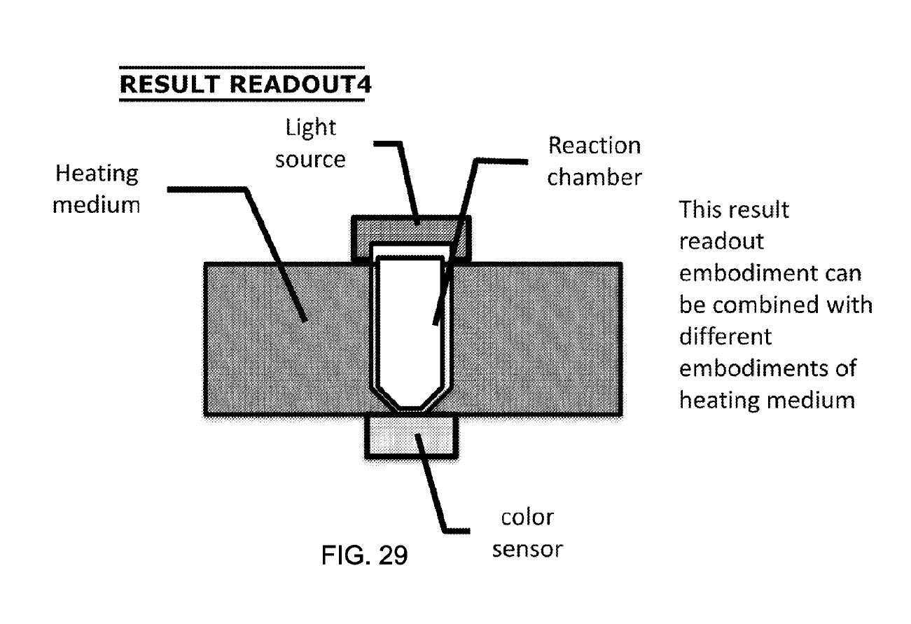

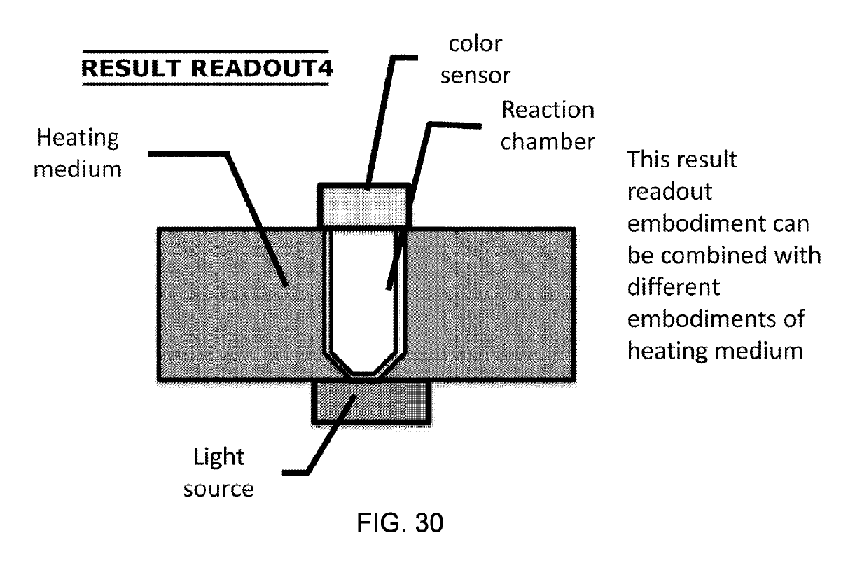

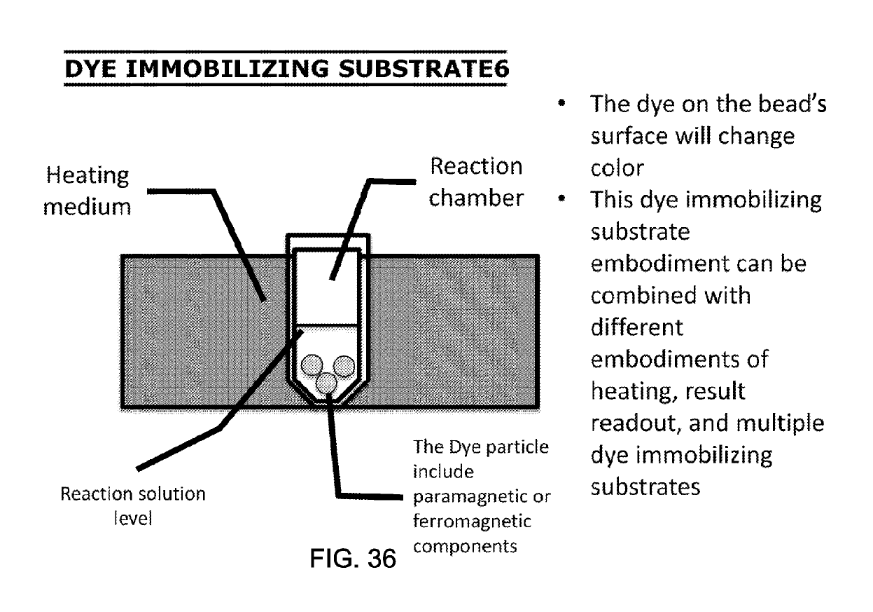

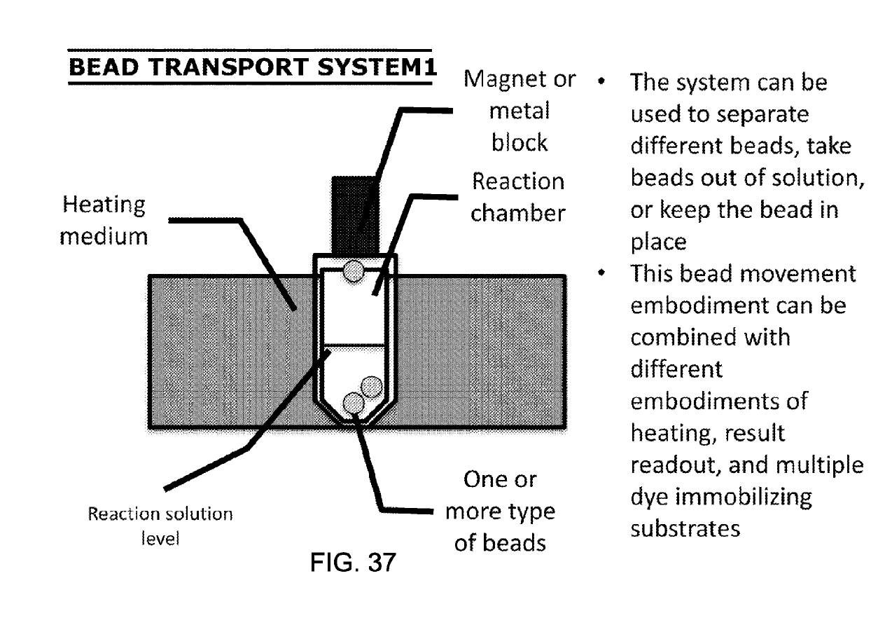

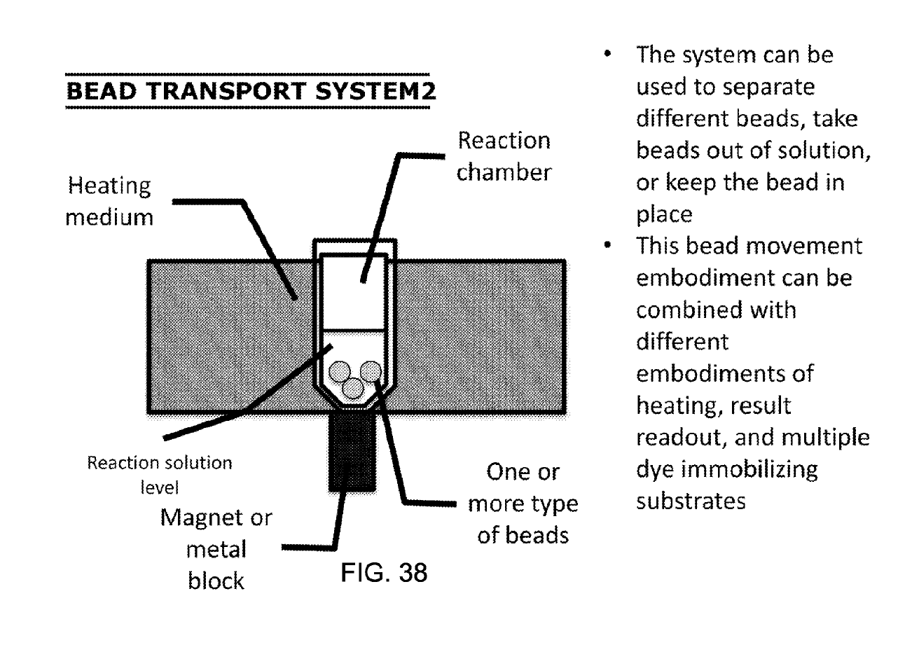

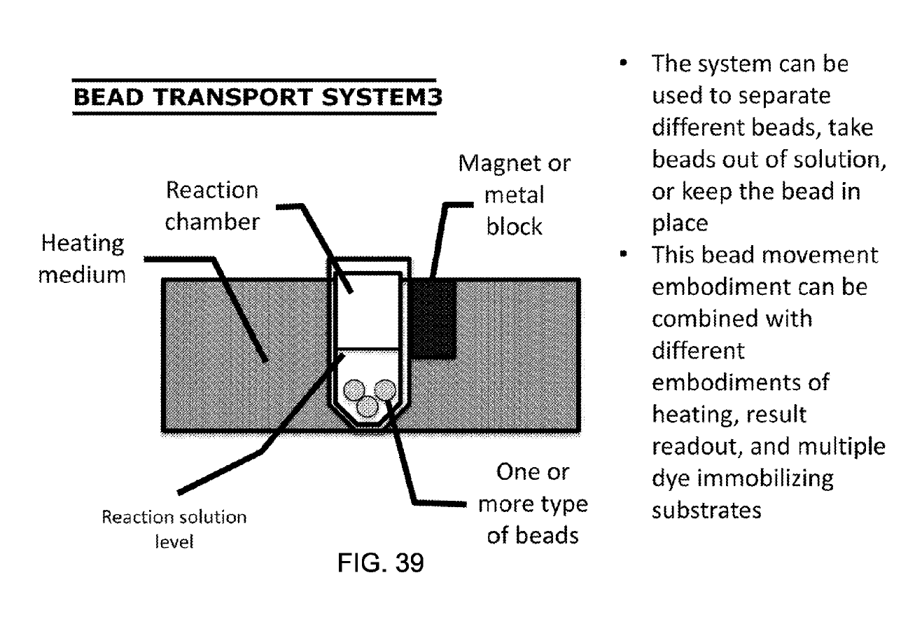

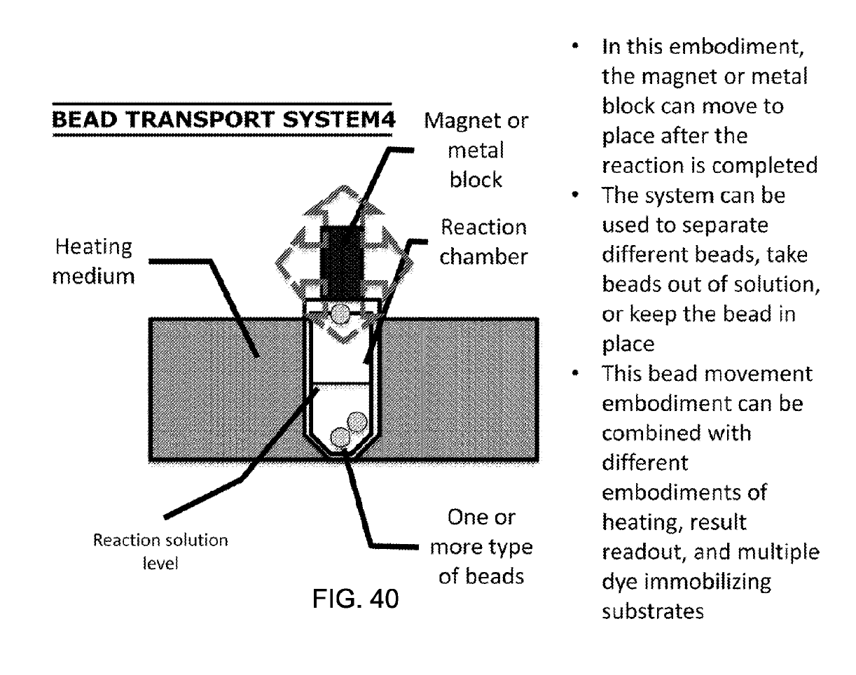

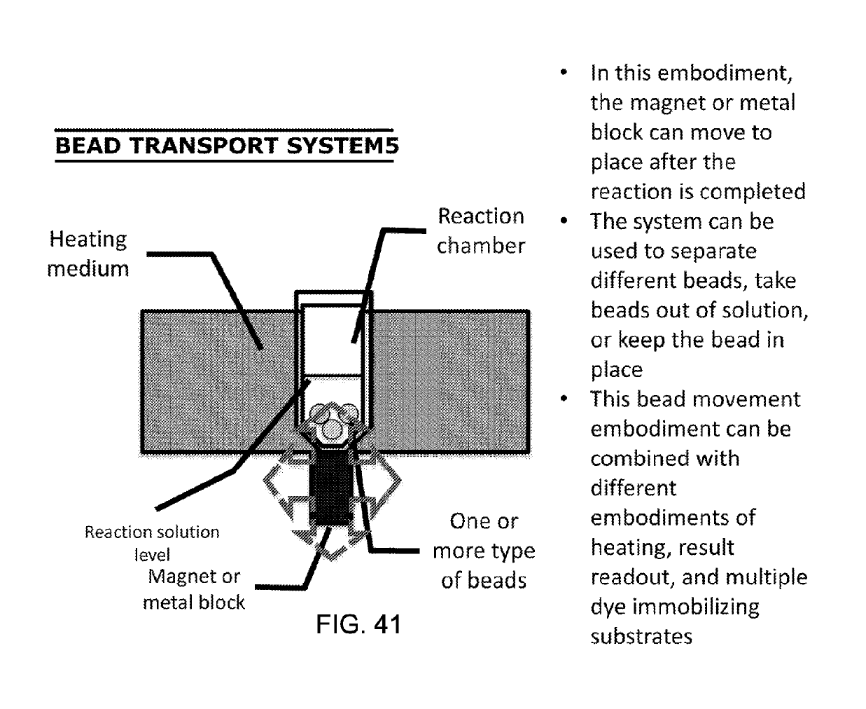

The present invention also relates to a device and/or a machine (together referred to as a device) provided for analyzing biological reactions such as nucleic acids (see FIGS. 24-42). This device may be part of the kit of the present invention. The device comprises a component for inserting at least one reaction chamber. An electrical sensor and display unit may also be included for electrical readout and displaying the result (see FIGS. 26, 27, 28 and 29). The device can further comprise at least a status light to indicate the status of the reaction. Additionally, at least one heating element container (see FIG. 25) is provided and/or mechanism to control thermal heating and/or cooling. The device of the invention has a readout component, either an opening for reading or light guide. If a light guide is part of the device, a light source and colour sensor are added to the device (see FIGS. 29 and 30). The position of the light source and/or colour can vary. Furthermore, a display unit and/or display lights are found on the device in order to provide instructions to the user and/or display the results.

The device of the present invention also can include a magnet component. The magnet is located either in a separate chamber or container (vessel, vial, test tube, tube chamber, or container used interchangeably, herein) from the container containing the sample, or the magnet can be in the form of beads found in the same container as the sample and reaction. In the event the magnet is found in a separate container, transferring the sample and/or reaction resultant sample to the container can be done for instance, by pipetting the sample into the magnet-container or the use of other transfer means also can do so.

In the situation where the reactions are used for testing, amplification and other reactions where infectious diseases are involved, it may be preferable to utilize the device that maintains the magnets in the same container, thereby avoiding the opportunity for contamination.

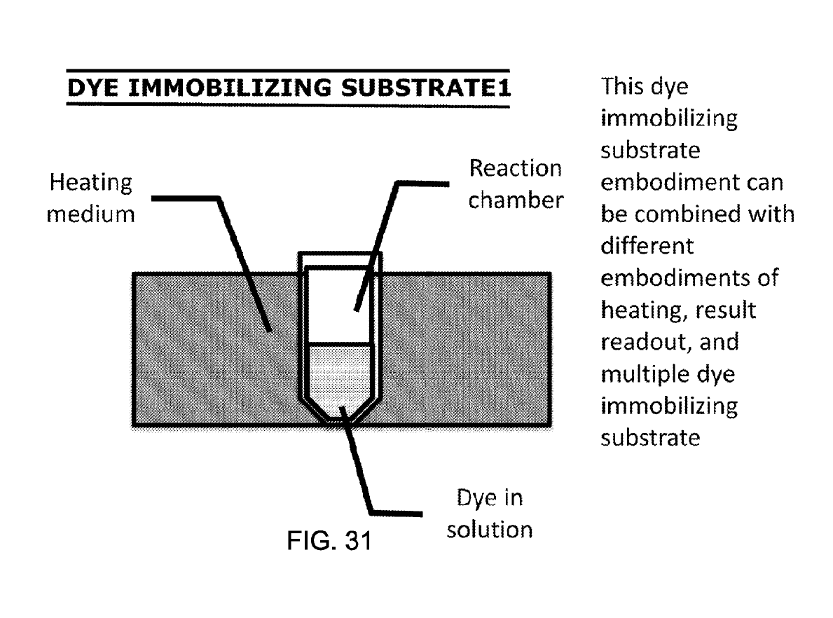

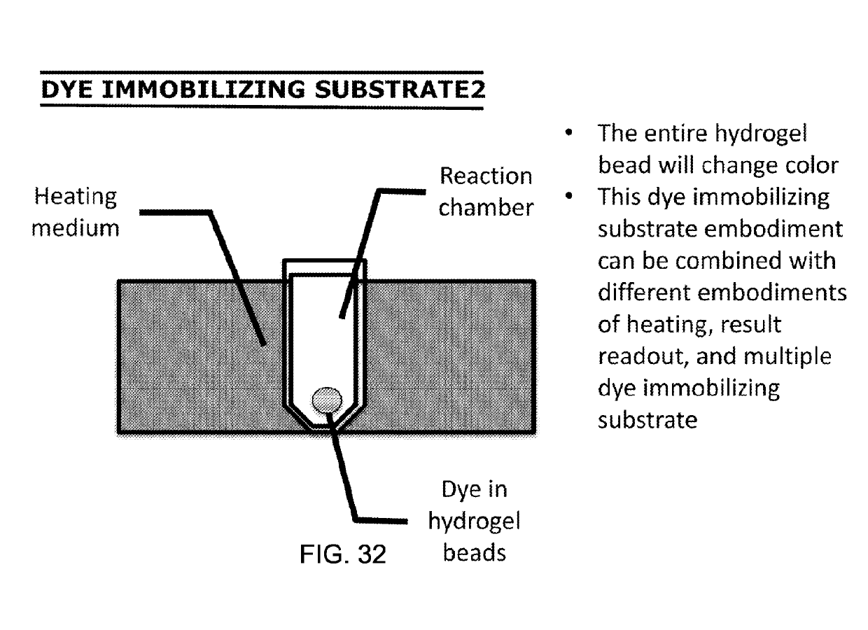

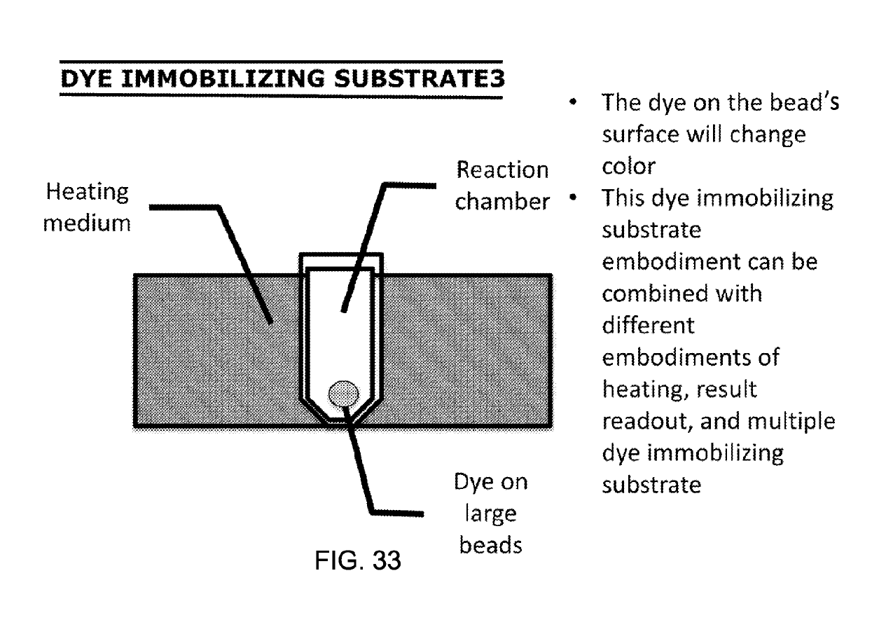

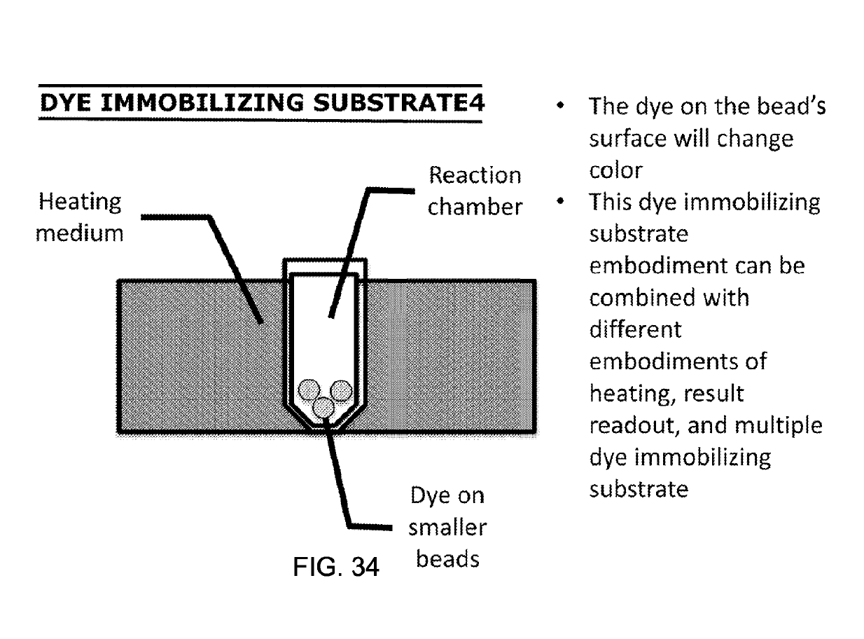

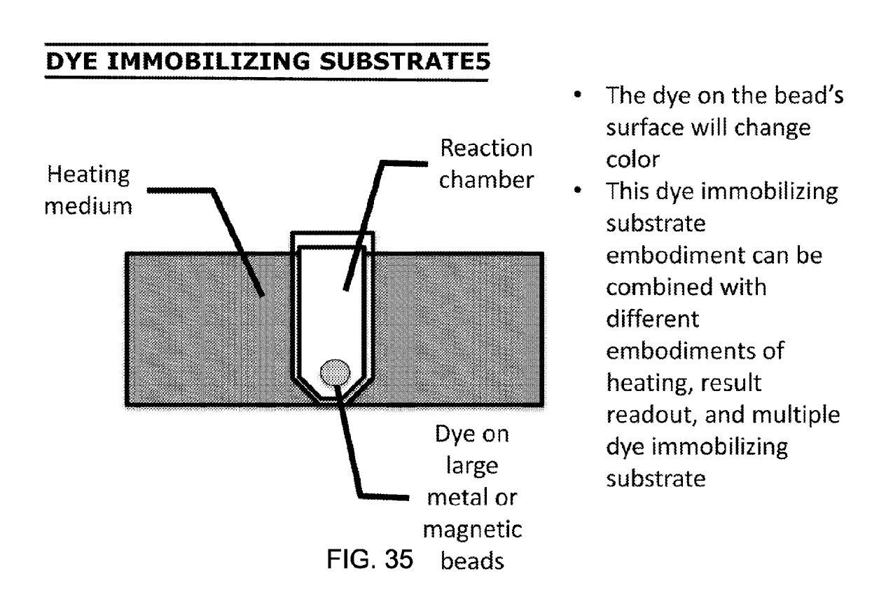

In the event a dye kit is provided in the device, a receptacle for that solution is configured into the reaction chamber of the device (see FIG. 31). As indicated herein, the dye used in the present invention can be placed on any size bead (see FIGS. 32 and 33). Also, the beads may be metal or magnetic, wherein a magnet or metal box may be made part of the device, at various locations in order to accomplish fixation of the beads and/or transporting the bead location (see FIGS. 34-41).

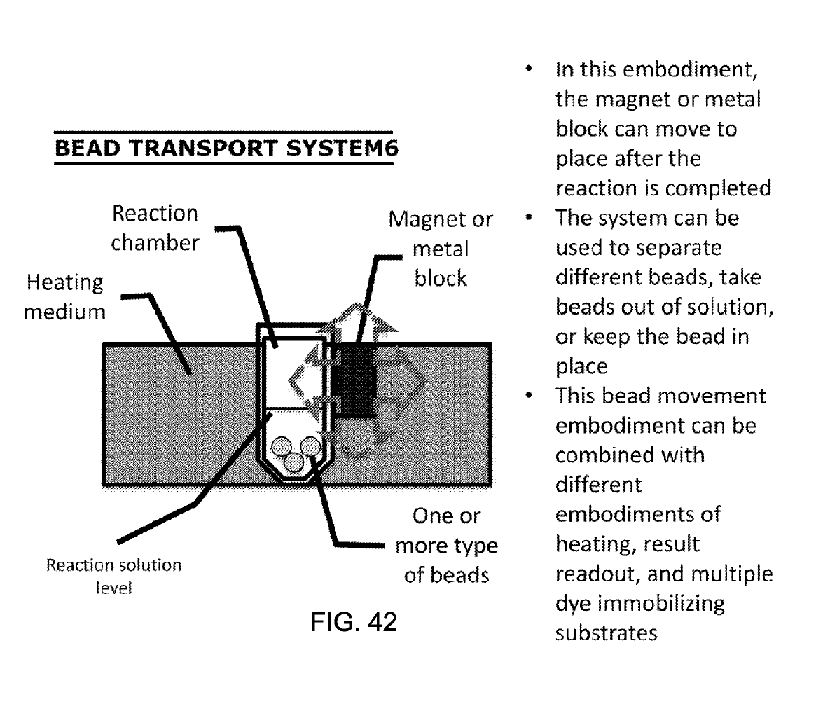

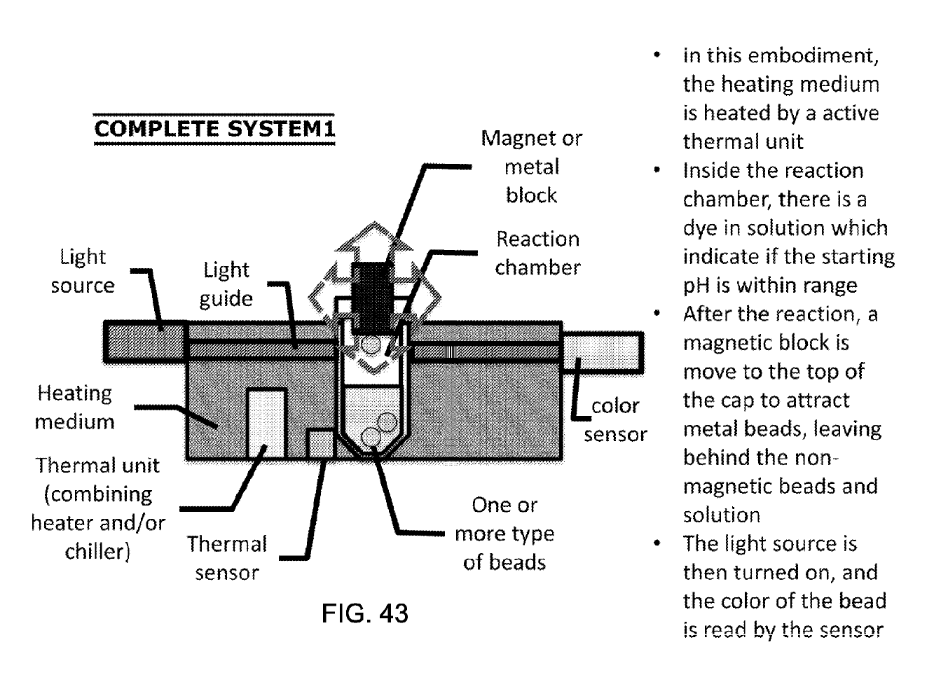

Note that the heating mechanism sensor light source, light guide and colour sensor can all be part of the device of the present invention (see FIG. 42). The light source and colour sensor can be located and guided through the same light guide.

The device of the present invention can be used by connecting it through a data port to a telephone or computer for readout of the results.

Chemical conjugation to an organic polymer, minimal scaffold, e.g. silica, alumina oxide, is well established in the art. When using the polymer or minimal oxides, the material can be molded, heat formed, coagulation formed, or printed. These material could be engineered into desired form and shapes. The material in the kit can be a film or bead or integrated as part of the reaction container, such as being printed on the reaction containers. In the event printing is used, the material can be printed to form a pattern, that can be a mix of text, mark, symbol sign, or any chosen form. The background where the conjugated dye is printed can have the same or a different colour to facilitate the recognition of the reaction result. For example, the colour of the deprotonated K2 dye is magenta. When the K2 dye is printed as a plus sign, “+”, on a magenta background, the sign is only recognizable when the K2 dye is gradually protonated.

Cellulose acetate is a particular preferred material for pH dye conjugation. The material is enzyme friendly, and the cost to use it in a kit is very low. The physical and chemical properties do not alter the enzyme with respect to the pH sensing application. Hydration is very rapid, and the physical or chemical properties do not change much between wet and dry states. Cellulose acetate also can be molded into various shapes. It is also the available in the form of a thin film. The surface can be rendered glossy and reduce the number of pores. For bead mass manufacturing, cross-linked bead can be formed by coagulation, e.g. U.S. Pat. No. 5,972,507, incorporated herein in its entirety by reference.

In the kit of the invention, the pH dye is linked to the cellulose acetate. The K2 indicator is one example of an activated pH indicator dye that can be conjugated with an enzyme friendly material, such as cellulose acetate. The present invention is not limited to the K2 dye or cellulose acetate.

Fabricated nanoliter reactor chambers in silicon with integrated actuators (heaters) for PCR monitoring exist, see for example Iordanov et al. ‘Sensorised nanoliter reactor chamber for DNA multiplication, IEEE (2004) 229-232 (incorporated herein by reference in its entirety). As noted by Iordanov et al. in the above-noted paper, untreated silicon and standard silicon-related materials are inhibitors of Taq polymerase. Therefore, when silicon or a silicon-related material, e.g. silicon germanium or stained silicon (hereinafter “silicon”) is employed for fabrication of the chamber or channel for nucleic acid amplification, it will usually be covered with material to prevent reduction of polymerase efficiency by the silicon, such as SUS, polymethyl-methacrylate (PMMA), Perspex™ or glass.

Microfabricated silicon-glass chips for PCR are also described by Shoffner et al. In Nucleic Acid Res. (1996) 24, 375-379 incorporated herein by reference in its entirety. Silicon chips are fabricated using standard photolithographic procedures and etched to a depth of 115 μm. Pyrex™ glass covers are placed on top of each silicon chip and the silicon and glass are bonded. These are but a few examples of surfaces for use in the present invention. Others include oxidized silicon.

As an alternative, the sample for PCR monitoring may flow through a channel or chamber of a microfluidic device. Thus, for example, the sample may flow through a channel or chamber which passes consecutively through different temperature zones suitable for the PCR stages of denaturing, primer annealing and primer extension.

Thus, in one embodiment for the present kits, the sample for nucleic acid amplification flows through a microfluidic channel on a substrate, and as it flows it consecutively passes through temperature zones provided in the substrate or base suitable for successive repeats along the length of the channel. The pH indicator dye can be incorporated in all the PCR embodiment described herein above.

While the above illustrates generally a kit for a PCR system designed to achieve thermo-cycling, various isothermic nucleic acid amplification techniques are known, e.g., single strand displacement amplification (SDA), and DNA or RNA amplification using such techniques may equally be monitored in accordance with the invention.

All primers used in the present examples are synthesized by Integrated DNA Technologies or Thermo Fisher. As the presence of the pH dye in the reaction causes minimal effect on the amplification reaction, there is no need to change the composition of the amplification reagents. The only exception is that the magnesium ion (Mg2+) should be high enough, e.g. 1.5 mM or preferably 2 mM or higher, such that deoxynucleotides form complexes with the magnesium ion.

LAMP is a process of amplification of double-stranded DNA that use primers in order to hybridize to the DNA and in order to target a specific sequence of interest. The amplification is achieved by primers forming hybridization with the template DNA extension from the inner primer which is later replaced by an outer primer by the strand-displacement activity of the polymerase and the exponential amplification of the target sequence and the newly synthesized strands.

The primer, deoxynucleotides (dNTPs), reaction buffer, indicator dye, and polymerase are premixed without particular order of the step, apart from the polymerase which is added in the last step to prevent non-specific reaction. For reactions that use non-lyophilised formulation, the reagents above should be assembled on a chilled box to prevent non-specific reaction. The sample DNA, such as purified human genomic DNA, fresh human whole blood, lambda DNA, pUC19 plasmid, or any other nucleic acid template is added at the last step before sealing the container and putting the container to a heat block, if heating is required. At the end point of the amplification reaction, the reaction container is observed by the un-aided eye or by a simple camera.

When lyophilised reagent is used, the first step is to re-suspend the dried reagent with water before adding the sample target. The rest of the steps follow the same order described in the non-lyophilised reaction.

In a typical immobilization procedure using the kit of the present invention, 100 mg of the finely grounded indicator dye is mixed with 1 g concentrated sulfuric acid and left for 30 min at room temperature for conjugation preparation. The mixture is diluted into 900 ml water and 1.6 ml 32% w/v sodium hydroxide solution. Then, 100 mL of 25% w/v sodium carbonate followed by another 5.3 ml of 32% w/v sodium hydroxide solution are added. At this stage, the enzyme-friendly surface, e.g. cellulose acetate, is mixed with the dyeing solution. The sulfonate of the dye is converted into a reactive vinylsulfonyl derivative, that allows a Michael addition reaction with the reactive groups of the enzyme-friendly surface. The reaction time depends on the porosity of the material and the thickness of the conjugated layer required. After reaction, the unconjugated dye is removed by excessive rinsing with water. The conjugated dye with the enzyme-friendly surface can be then stored dried or in water solution. The conjugated dye is then ready for the nucleotide identification kit.

As described previously, it is preferred to physically separate one nucleotide reaction from another to prevent interference. In other examples, where amplification of nucleic acid is involved, e.g. LAMP or PCR, the reaction container is typically sealed during and after the interrogation reaction. Sealed containers will prevent any aerosol of the product from contaminating other reactions.

The kit containing the conjugated dye contains a reagent with which it is mixed as a discrete object or integrated into the surface of the container. The reaction is then monitored and detected by the colour change of the conjugated dye.

The kits of the present invention do not require instruments for nucleotide identification or pH changes. Performing nucleotide identification is simple with a kit of the present invention comprising the dye kit, nucleic acid amplification reagent, e.g. dNTP and polymerase, a container to hold the reaction, and a heat source to provide the reaction condition. In one form, the reaction container is placed in a heat block. In another form, the nucleotide identification is carried out by putting the reaction container in a hot water bath, which could be replaced after each test to avoid contamination. The choice of the water bath depends on the number of reactions and environment of the test. A simple cup warmer in an office and a glass of water is sufficient to provide the heating for nucleotide identification using isothermal reaction. As the examples described herein, the complexity of the nucleotide identification an be designed to satisfy the restriction on the training of the user. The invention is not restricted to the heating methods described herein. Other heating methods include heating by radiation, such as microwave oven, infrared laser, infrared lamp, or solar energy.

It is not unusual that nucleotide identification involves a sensor and/or a circuit to provide the result of the measurement. In one example, the colour is recognized by a camera, and the change of the colour is monitored. The rate of the colour change is a function of the reaction rate, which is a function of the sample amount or copy number.

The present invention is particularly suited to detection and measurement of pH methods as discussed and disclosed in U.S. patent Ser. No. 13/618,694, incorporated herein by reference in its entirety. The present invention, for instance, is useful for targeting proteins in chemical and/or biological reactions such as, but not limited to, ELISA reactions.

The objects of the present invention are further described by the following examples provided as illustrative of the present invention and not limited thereof.

As an example, indicator dye, 4-[4-(2-Hydroxyethanesulfonyl)-phenylazo]-2,6-dimethoxyphenol is immobilized on cellulose acetate beads. The size of the bead is 2 mm in diameter. The pKa of the dye after immobilization is around 7.5. When the bead is mixed with the 50 micro litre of LAMP reaction mixture (50 mM potassium chloride, 5 mM magnesium sulfate, 5 mM ammonium chloride, 0.1% w/v tween 20, 1M betaine, 2 mM deoxynucleotides, 32 U Bst polymerase, 1 mg/mL bovine serum albumin, 1000 copies of lambda DNA, 1.6 micro M, lambda_FIP primer and lambda_BIP, 0.8 micro M lambda LF and lambda LB primer, 0.2 uM lambda_F3 and lambda_B3 primer, pH 8.5, the colour of the bead is deep magenta. The beads are mixed with all the LAMP reagents and sealed in a micro tube. Two replicates are performed. After the enzyme reaction (63° C. for 45 minutes), the pH change from the enzyme reaction is visually recognizable as bright yellow (FIG. 1).

### Example 1

### Detection of Nucleic Acid Amplification Using Kit of the Present Invention

In FIG. 1, the colour of each dye film corresponds to a pH range. (FIG. 1)

The K1 film is a cellulose film of 20 micrometer thickness conjugated with potassium 1-hydroxyl-4-[4-(hydroxyethylsulphonyl)-phenylazo]-naphthalene-2-sulphonate.

The K2 film is a cellulose film of 20 micrometer thickness conjugated with 4-[4-(2-hydroxylethanesulfonyl)-phenylazo]-2,6-dimethoxyphenol

The K1 solution is potassium 1-hydroxyl-4-[4-(hydroxyethylsulphonyl)-phenylazo]-naphthalene-2-sulphonate

The K1 particles are Cellulose Microparticles Avicel® PH-101. 50 micrometer in diameter is conjugated with potassium 1-hydroxyl-4-[4-(hydroxyethylsulphonyl)-phenylazo]-naphthalene-2-sulphonate

Detection using different dye forms:

Three different forms of K1 dye are used in the assay, K1 film, K1 particle, and soluble K1. The assay shows the compatibility of the dye form and the LAMP reaction. The LAMP reactions are set up to use p450 2C19 wild type primer set and K562 genomic DNA. 1 ng of K562 which is about 300 copies is mixed with the reaction components. Dye is included in each tube before the reaction. The reaction is held at 63 degree Celsius for 30 mins, and the colour of the reaction is observed.

| | Final concentration | |
|---|---|---|
| Primers mix solution | | |
| 2C19_FIP.Wild | 1.6 | uM |
| 2C19_BIP.Wild | 1.6 | uM |
| 2C19_LF | 0.8 | uM |
| 2C19_LB | 0.8 | uM |
| 2C19_F3 | 0.2 | uM |
| 2C19_B3 | 0.2 | uM |
| Mutant primers mix | | |
| solution | | |
| 2C19_FIP.Mut | 1.6 | uM |
| 2C19_BIP.Mut | 1.6 | uM |
| 2C19_LF | 0.8 | uM |
| 2C19_LB | 0.8 | uM |
| 2C19_F3 | 0.2 | uM |
| 2C19_B3 | 0.2 | uM |
| LAMP buffer | | |
| KCl | 50 | mM |
| MgSO4 | 5 | mM |
| NH₄Cl | 5 | mM |
| BSA | 1 | mg/mL |
| Tween 20 | 0.10% | |
| Betain | 1M | |
| Deoxynucleotides | 2.8 | mM |
| Bst polymerase | 32 | U |
| H2O | Fill to 50 | uL |

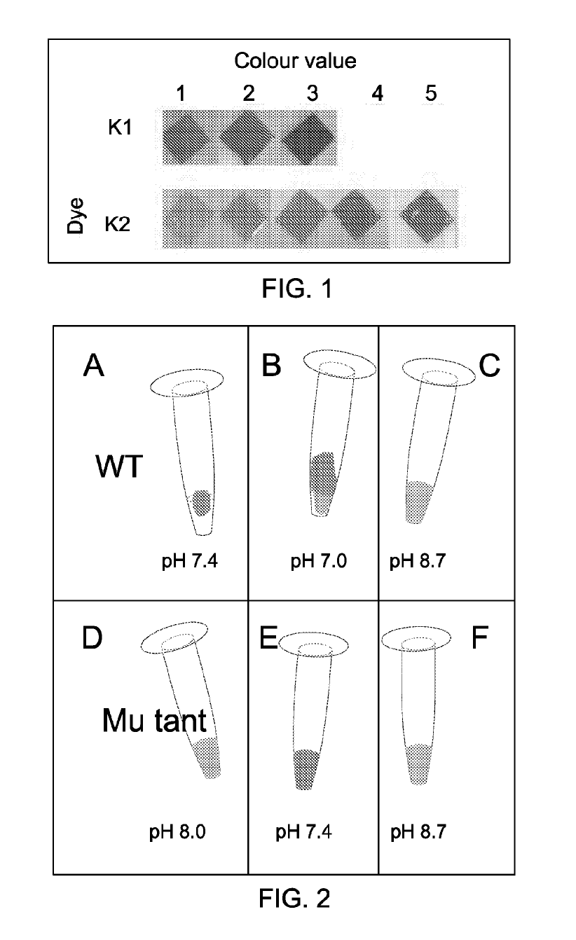

The photo in FIG. 2 shows the colour response of the pH film in LAMP reaction for 2C19 genotyping. The photo is taken after the LAMP reaction. In the graph the order are K1 film wildtype (A) or mutant (D); K1 powder wildtype (B) or mutant (E); K1 solution wildtype (C) or mutant (F).

TABLE 1

| | | Before reaction | | After reaction | |
|---|---|---|---|---|---|
| | | | Colour | | Colour |
| | | pH value | value | pH value | value |
| Positive control | No dye | 8.7 | 0 | 6.4 | 0 |
| No template | No dye | 8.7 | 0 | 7.8 | 0 |
| control | | | | | |
| 2C19 Wildtype | K1 Film | 8.7 | 3 | 7 | 1 |
| | K1 Powder | 8.7 | 3 | 7.4 | 1 |
| | (1 mg) | | | | |
| | Soluble K1 | 8.7 | 3 | 8.7 | 3 |
| 2C19 Mutant | K1 Film | 8.7 | 3 | 7.4 | 1 |
| | K1 Powder | 8.7 | 3 | 8 | 3 |
| | (1 mg) | | | | |
| | Soluble K1 | 8.7 | 3 | 8.7 | 3 |

The result of the colour value and the pH value of the K1 dye in the LAMP reaction are provided in Table 1.

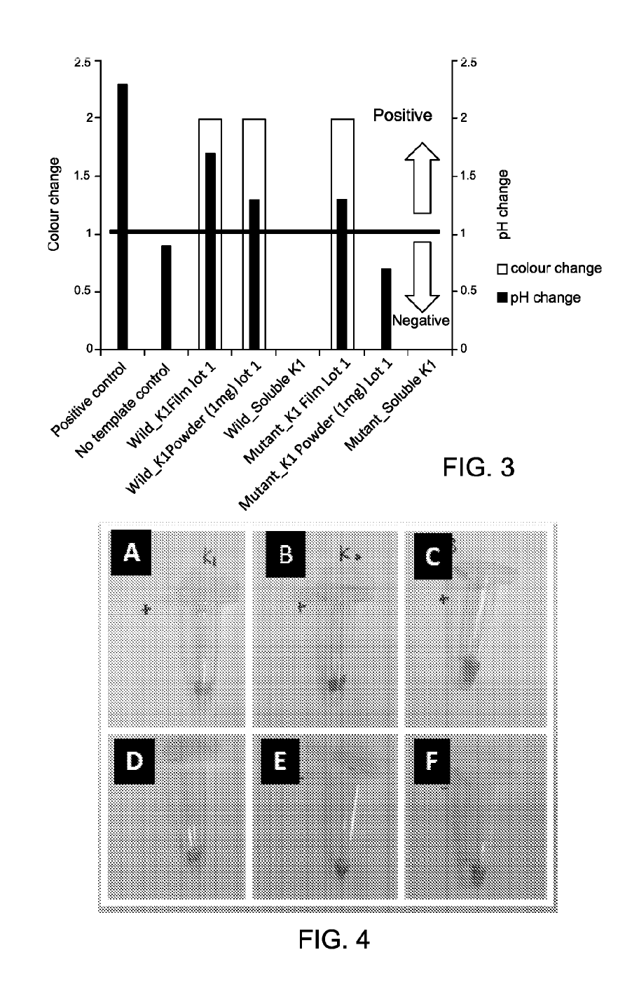

In FIG. 3, the K1 chemical is tested in the form of film, cellulose particles, and soluble molecules. (See above graph). The colour change of the 2C19 genotyping is converted into numbers using the colour panel. The chart shows the pH value change (Starting pH-end pH) and the colour change (starting colour-end-colour). When the threshold is held at 1 for colour change or for pH change, the sample with LAMP reaction is distinct from the one without the LAMP reaction. The pH value change is 100% in agreement with the colour change (See FIG. 3).

### Example 2

### Detection Using Different Dye Film and Chemicals

The reactions are set up to use p450 2C19 wild type primer set and K562 genomic DNA. 1 ng of K562 which is about 300 copies mixed with these reaction components. pH indicator dye is included in each tube before the reaction. The dNTPs is replaced by a 2.8 mM mixture of (deoxyadenosine triphosphate, deoxyguanosine triphosphate, deoxycytidine triphosphate) in the negative control samples. The reaction is held at 63 degree Celsius for 30 mins and the colour of reaction is observed.

| | Final concentration | |
|---|---|---|
| Wild type primers mix | | |
| solution | | |
| 2C19_FIP.Wild | 1.6 | uM |
| 2C19_BIP.Wild | 1.6 | uM |
| 2C19_LF | 0.8 | uM |
| 2C19_LB | 0.8 | uM |
| 2C19_F3 | 0.2 | uM |
| 2C19_B3 | 0.2 | uM |
| LAMP buffer | | |
| KCl | 50 | mM |
| MgSO4 | 5 | mM |
| NH₄Cl | 5 | mM |
| BSA | 1 | mg/mL |
| Tween 20 | 0.10% | |
| Betain | 1M | |
| Deoxynucleotides | 2.8 | mM |
| Bst polymerase | 32 | U |
| H2O | Fill to 50 | uL |

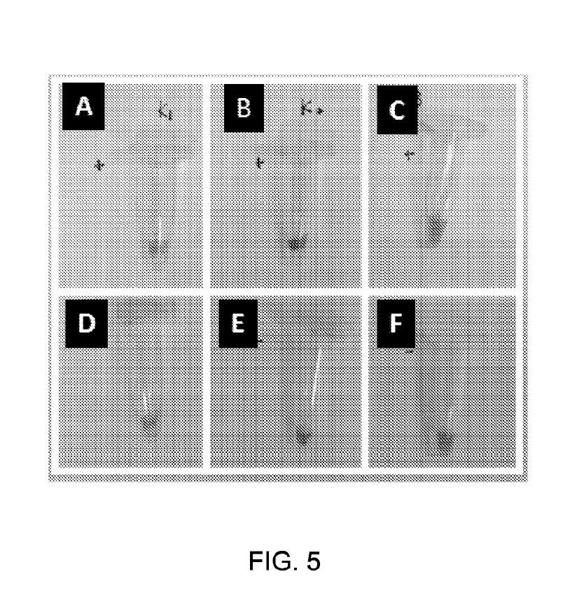

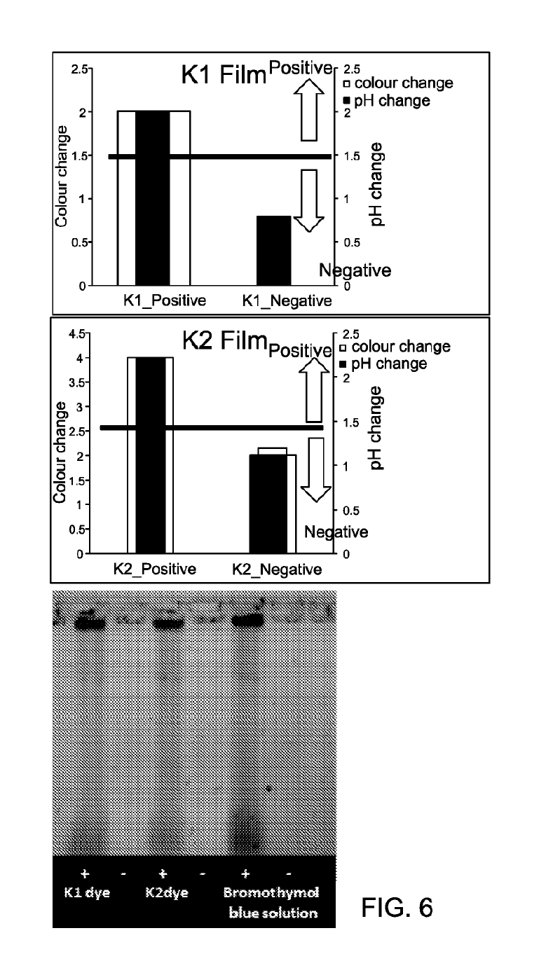

In each tube, a distinct dye film (K1 and K2) or a soluble pH indicator (bromothymol blue, 0.1 mg/mL) is mixed with the amplification reagents before the LAMP reaction. Two distinct films are tested for amplification detection. The photos of the reaction set up and results are shown in FIGS. 4 and 5. The colour changes are converted into valves by use of the coding panel found in FIG. 1. The compiled values are shown in Table 2 below. To visualize the colour difference and amplification versus no amplification, the value are plotted in FIG. 6. The result shows colour changes in the presence of the template.

The photo of FIG. 4 shows the colour of the dye in each tube before the LAMP reaction. In the photo the tubes are K1 film LAMP reaction with template (A) and without template (D), K2 film with template (B) and without template (E), bromothymol blue solution with template (C) and without template (F).

The photo of FIG. 5 shows the colour of the dye changed in the tube where amplification occurs in the LAMP reaction (top row) while the colour of the dye remained unchanged where there is no amplification in the LAMP reaction (bottom row). In the photo the tubes are K1 film LAMP reaction with template (A) and without template (D), K2 film with template (B) and without template (E), bromothymol blue solution with template (C) and without template (F) (See FIG. 5).

TABLE 2

| | | Before | | After | |
|---|---|---|---|---|---|
| | | LAMP | | LAMP | |
| | Lamp | | Colour | | Colour |
| | reaction | pH value | value | pH value | value |
| K-1 | Yes | 8.7 | 3 | 6.7 | 1 |
| | No | 8.7 | 3 | 7.9 | 3 |
| K-2 | Yes | 8.7 | 5 | 6.5 | 1 |
| | No | 8.7 | 5 | 7.5 | 3 |
| BB | Yes | 8.7 | Blue | 6.6 | Blue |
| | No | 8.7 | Yellow | 7.8 | Yellow |

The table 2 above is of the colour result and the pH correlation to the LAMP reaction.

Two distinct films are tested for amplification detection in FIG. 6. Two distinct pH indicators are immobilised on cellulose films. The colour change of each film is converted into a number using its own colour panel. The chart shows the pH value change (Starting pH-end pH) and the colour change (starting colour-end colour). The value of LAMP reactions is distinctly differentiate from the one without the LAMP reactions in all three dye films. The pH value change is 100% in agreement with the colour change. The sample from each tube is analyzed using agarose electrophoresis in FIG. 6.

The intensity of the colour change is very strong such that the result could easily be determined by the un-aided eyes. Significant colour change is also present when a soluble dye (bromothymol blue, 0.1 mg/mL) is used as an indicator. It shows that it is possible to use soluble dye.

However, at higher concentration, the dye inhibits the reaction. A similarity is also observed when a soluble K1 dye is mixed with the LAMP reaction. The soluble chemical is prone to interfere and inhibit the amplification.

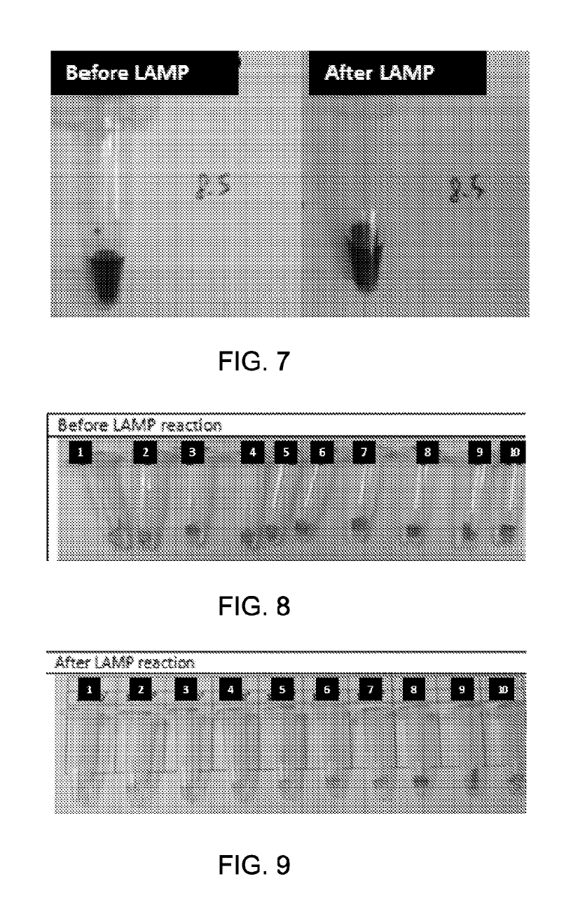

The bromothysial blue did not produce a colour change and the pH remains unchanged at 8.5 (See FIG. 7).

### Example 3

### Detection of Nucleic Acid at Low Concentration

The reactions are set up to use lambda primer set and lambda genomic DNA. The DNA template is diluted into various concentration that represent from 1, 10, 100, 1,000, 10,000, 100,000, 1,000,000, and 10,000000 copies of lambda DNA. K2 film is included in each tube before the reaction. The negative control does not contain lambda DNA. The reaction is held at 63 degree Celsius for 30 mins and the colour of the reaction is observed. The K2 film changes colour from deep magenta to bright yellow when there is amplification. The limit of sensitive shows in this assay is at 10 copies.

| | Final concentration | |
|---|---|---|
| Primers mix solution | | |
| Lambda_FIP | 1.6 | uM |
| Lambda_BIP | 1.6 | uM |
| Lambda_LF | 0.8 | uM |
| Lambda_LB | 0.8 | uM |
| Lambda_F3 | 0.2 | uM |
| Lambda_B3 | 0.2 | uM |
| LAMP buffer | | |
| KCl | 50 | mM |
| MgSO4 | 5 | mM |
| NH₄Cl | 5 | mM |
| BSA | 1 | mg/mL |
| Tween 20 | 0.10% | |
| Betain | 1M | |
| Deoxynucleotides | 2.8 | mM |
| Bst polymerase | 32.4 | U |
| H2O | Fill to 50 | uL |

The dye colour of each tube is pink before the LAMP reaction is found in FIG. 8. Each tube corresponds to a lambda DNA concentration (See FIG. 8).

FIG. 9 provides the dye colour changes to yellow for tubes 1 to 7. Tubes 8 to 10 remain pink. The result suggests the limit of detection is 10 copies of lambda DNA (See FIG. 9).

TABLE 3

| | | Before reaction | | | |
|---|---|---|---|---|---|
| Tube | Template | | Colour | After reaction | |
| number | Copies | pH value | value | pH value | Colour value |
| 1 | 1 × 10⁷ | 8.16 | 5 | 6.30 | 1 |
| 2 | 1 × 10⁶ | 8.16 | 5 | 6.43 | 1 |
| 3 | 1 × 10⁵ | 8.16 | 5 | 6.65 | 1 |
| 4 | 1 × 10⁴ | 8.16 | 5 | 6.66 | 1 |
| 5 | 1 × 10³ | 8.16 | 5 | 6.85 | 1 |
| 6 | 1 × 10² | 8.16 | 5 | 6.86 | 1 |
| 7 | 1 × 10¹ | 8.16 | 5 | 6.88 | 1 |
| 8 | 1 | 8.16 | 5 | 7.30 | 4 |
| 9 | No template | 8.16 | 5 | 7.40 | 4 |
| 10 | No template | 8.16 | 5 | 7.49 | 4 |

The results of the colour value and the pH value of the reactions of differing copy numbers is provided.

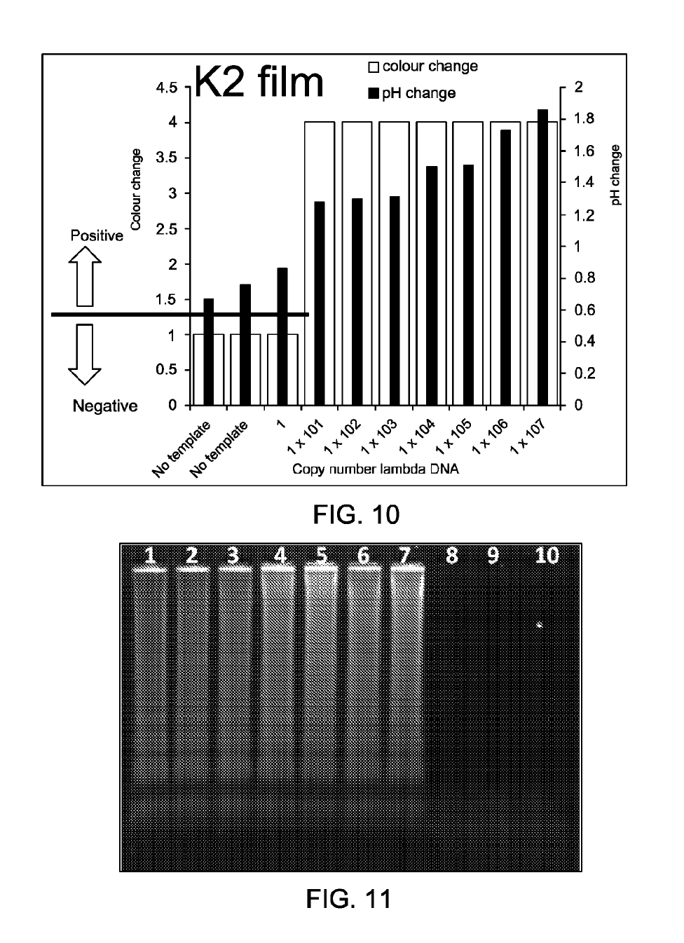

The chart in FIG. 10 shows that the discrimination of positive and negative response is easily differentiated. The detection using K2 film shows as low as 10 copies of lambda DNA are provided (See FIG. 10).

FIG. 11 provides the agarose electrophoresis photo showing that LAMP amplification occurs with lane 1 to lane 7, where the copy number is 10,000,000, 1,000,000, 100,000, 10,000, 1,000, 100, and 10 respectively. Lane 8 is corresponding to a single copy of lambda DNA where there is not amplification observed. Lane 9 and 10 are reaction without lambda DNA.

### Example 4

### Detection Under Murky Solution Such as Whole Blood

In each tube, a soluble pH indicator (bromothymol blue, 0.1 mg/mL), K1 film and a pH testing paper (Merck Millipore cat#1.09543.0001, non-bleeding paper) is mixed with the amplification reagents before the LAMP reaction

The reactions are set up to use p450 2C19 wild type primer set and K562 genomic DNA. 1 ng of K562 which is about 300 copies mixed with reaction components, 50 mM KCl, 5 mM MgSO4, 5 mM NH4Cl, 1 M betaine, 1 mg/mL BSA, 0.1% Tween 20, 2.8 mM dNTPs (deoxyadenosine triphosphate, deoxythymidine triphosphate, deoxyguanosine triphosphate, and deoxycytidine triphosphate), 1.6 microM FIP and BIP, 0.8 microM Loop-F and Loop-B, 0.2 microM F3 and B3, and 32 U of Bst polymerase in 50 uL reaction. The pH is adjusted to 8.0 before adding Bst, K562, or whole blood) The dNTPs is replaced by a 2.8 mM mixture of (deoxyadenosine triphosphate, deoxyguanosine triphosphate, deoxycytidine triphosphate) in the negative control samples. Then, 2 micro litre of fresh whole blood from a finger prick is added into each tube. The reaction is held at 63 degree Celsius for 30 mins and the colour of reaction is observed.

It is very challenging to see the difference between amplification versus no amplification in the presence of the whole blood in all except the K1 film. As it is simple and easy to remove the cloudy whole blood solution from the K1 film, the nucleic acid amplification is monitored as shown in the photos.

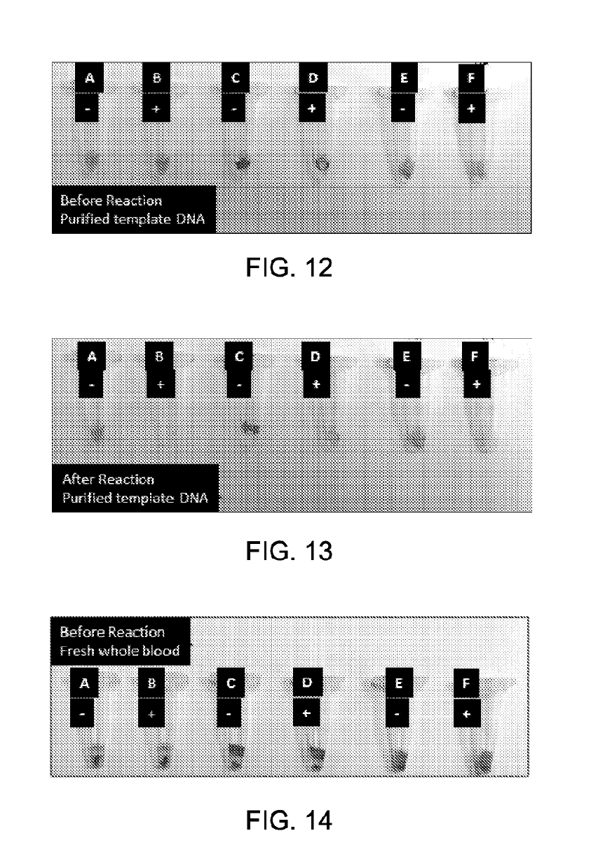

The photo in FIG. 12 shows the dye colour before the reactions that are with (positive) or without (negative) purified DNA. The reaction uses purified DNA as the template. From the left to right, the tubes contain the dye: bromothymol blue (A and B), K1 film (C and D), and pH testing paper (E and F). At pH 8 the tube A and B are in light blue, tube C and D (K1 film) are in deep magenta, and tube E and F (pH paper from Merck-Millipore) are in greenish brown.

The photo in FIG. 13 shows the dye colour after the reaction that is with (positive) or without (negative) the DNA template. The colour of the dye changed when there was DNA template in the reaction. The tube B (bromothymol blue) changes from light blue to yellow. The tube D (K1 film) changes from deep magenta to orange. The tube F (pH paper) change from greenish brown to bright yellow.

The photo in FIG. 14 shows the whole blood effect on dye colour before the reactions. The tubes with template DNA are labeled with positive signed while the tubes without added DNA are labeled with negative sign. Each tube contains 2 microlitre of fresh whole blood. From the left to right, the tubes contain bromothymol blue (A and B), K1 film (C and D), and pH testing paper (E and F). At pH 8 the bromothymol blue is in light blue, K1 film is in deep magenta, and pH paper from Merck-Millipore is difficult to define the colour due to the heterogeneous colour mix.

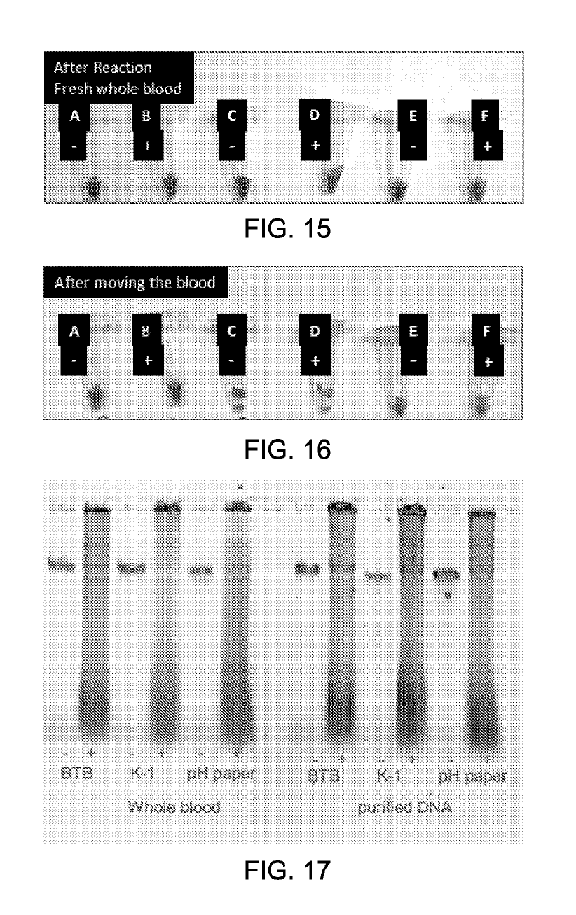

The photo in FIG. 15 shows the whole blood effect after the reactions. The colour of the soluble dye, bromothymol blue (A and B), becomes indistinguishable with the presence of the whole blood.

The photo in FIG. 16 shows the colour of the immobilised dye after shaking the solution off the dye. The blood could be removed from the immobilised dye in the case of K1 film (C and D) and the pH paper (E and F). The removing process does not require user to open the tube therefore there is not risk of contamination. After removing the blood, the colour of the pH paper is also difficult to differentiate amplification (F) from no amplification (E). This is due to the porous structure of the paper that has trapped the blood within. The colour of K1 film is the only reaction that shows distinct difference between the no amplification (C, colour value=3) and amplification (D, colour value=1)

FIG. 17 shows a LAMP reaction from each tube using agarose electrophoresis. BTB is bromothymol blue (See FIG. 17).

### Example 5

### PCR Embodiment

| | Final concentration | |
|---|---|---|
| Primers mix solution | | |
| HCV core Forward primer | 1 | uM |
| HCV core Reverse primer | 1 | uM |
| PCR buffer | | |
| KCl | 50 | mM |
| MgCl4 | 2 | mM |
| Deoxynucleotides | 1 | mM |
| Taq polymerase | 2.5 | U |
| H2O | Fill to 30 | uL |

The indicator dye film for monitoring the nucleic amplification is used in PCR. The film is compatible with the PCR reaction condition. In one example, the assay is assembled by using a plasmid containing a Hepatitis C virus core 1b gene. The reactions are setup with the dye film before the PCR reaction. The pH of each reaction is adjusted to between 8.0-8.2. The thermo-cycling programme follows an initial denaturation step at 94 degree Celsius for 2 minutes, with 55 repeats of three-step module: 94 degree Celsius for 30 seconds, 65 degree Celsius for 20 seconds, and 72 degree Celsius for 15 second. The reaction is finished holding the last step of the reaction at 72 degree Celsius for 2 minutes. The colour of the tubes is seen after they are taken out from the machine.

The result shows the distinct colour difference between tubes with amplification (yellow) and tubes without amplification (pink).

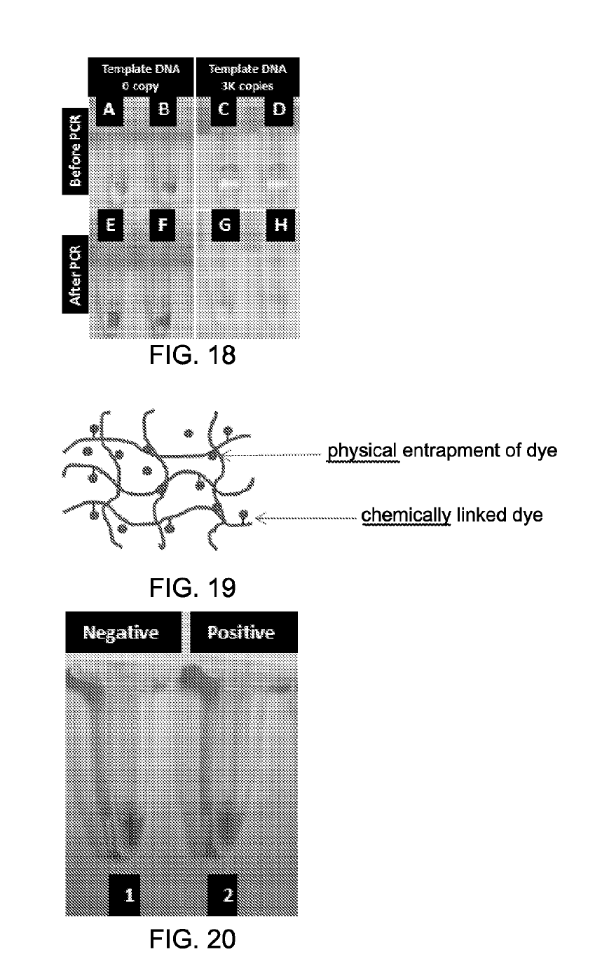

The result of the PCR reaction shown in FIG. 18 with the presence of the dye is provided. K1 films are shown in A, C, E, and G while K2 films are shown in B, D, F, and H. Before the PCR reaction, all films show orange. After the PCR reaction, the tubes without amplification (E and F) show pink. The tubes with plasmid templates where the amplification occurs show yellow (G and H).

### Example 6

### 3D Dye

### Hydrogel Assay

It has been a long felt wish the development of an assay that could detect a gene without sample preparation and without requiring more than 2 steps from sample to and result and without instruments for the result interpretation. The present invention provides a method that fulfills these requirements. Genes are amplified in the presence of whole blood directly from the finger prick. The presence of the gene is detected by monitoring the amplification using an immobilised dye. The results in reducing all these steps into one.

First, water is loaded from a predefined volume container to one or more reaction container(s) that contain lyophilised amplification reagents in the presence of the indicator dye and the sample is loaded, such as whole blood, into the reaction container(s).

To prevent contamination, the container should remain instrument remain securely closed after any nucleic acid amplification. Without the help of any instruments, the amplification result would usually be difficult to read, when the amplification reaction is not a clear solution, such as whole blood amplification. To overcome the interference from the suspended colloidal particles or the coloured compounds that come with the sample, the samples are usually pre-treated by dilution or heating or both. Examples cover the conventional detection without instrument such as DNA chelating fluorescence dye, YO-PRO-1 or Sybr Green (Genome Letters, 2, 119-126, 2003), metal chelating dye, Calcein and hydroxy naphthol blue (Biotechniques, 46, 167-172, 2009).

The present invention demonstrates that the dye chemicals (K1 and K2) are covalently linked to a hydrogel 3D object which fits into the container where the amplification occurs. It is shown from our disclosure using films that are conjugated with the K1 or K2, allow the unaided eyes to easily read the nucleic acid amplification result. However, without opening the reaction container, it is not always easy to separate the solution from the film in a container, as the film tends to stick to the wall of the container. The 3D object solves the problem by minimizing the contact surface between the indicator dye and the container.

The 3D object is a ball such that the contact area between the 3D object and the reaction is minimized. The 3D ball can be formed by applying a layer of hydrogel to a ball, such as polystyrene ball, cellulose ball, or ball made of other material. Different colours of the ball are selected to enhance the contrast of the indicator colour dye to facilitate even better colour change for the unaided eye.

The present invention also describes a design where the dye is an indicator ball or a 3D dye indicator object is influenced by an external magnetic field. When paramagnetic or ferromagnetic material is embedded in the 3D object or ball, it is possible to control the position of the dye such that the dye can be viewed without the interference of the cloudy solution and is done so with the container securely sealed. The embedding is as simple as punching an iron pin into a polymer ball before the hydrogel coating.

Yet in another embodiment, the 3D object is a collection of small particles that can form a cluster of 3D objects under the influence of an external magnetic force. The particles are of micro meter in diameter in equivalent to a spherical ball or other sizes that are reasonably easy for magnetic manipulation.

The hydrogel is made up of Poly(2-hydroxyethyl methacrylate) (PHEMA), Polyurethane (PU), Poly(ethylene glycol) (PEG), polyethylene glycol methacrylate (PEGMA), polyethylene glycol dimethacrylate (PEGDMA), polyethylene glycol diacrylate (PEGDA), Poly (vinyl alcohol) (PVA), Poly(vinyl pyrrolidone) (PVP), or Polyimide (PI).

The dye is any reactive vinylsulphonyl dye or pH indicator dye.

A hydrogel is formed by using poly(2-hydroxyethyl methacrylate), the hydrogel is conjugated with K2 dye, also known as 4-[4-(2-Hydroxyethanesulfonyl)-phenylazo]-2,6-dimethoxyphenol indicator dye (vinylsulphonyl dye)

### The Material are:

1) 2-hydroxyethyl methacrylate (HEMA), poly(ethylene glycol) dimethacrylate, 2,2-Dimethoxy-2-phenylacetophenone, 4-[4-(2-Hydroxyethanesulfonyl)-phenylazo]-2,6-dimethoxyphenol (pH indicator dye), Sulfuric acid, Sodium hydroxide, and Sodium carbonate

### Hydrogel Preparation

The Chemical composition of reagents used in the hydrogel are given in table 4.

| Reagent | Mass % |
|---|---|
| HEMA | 63 |
| poly(ethylene glycol) dimethacrylate | 1.5 |
| 2,2-Dimethoxy-2- | 0.5 |
| phenylaectophenone (DMPA) | |
| DI water | 35 |

Table 4: Chemical Composition of Reagents Used for Formation of Hydrogel

All the reagents are added together after weighing and subjected to stirring for 10 min to obtain a homogeneous mixture. This mixture is solvent casted into the glass petridish. The petridish is subjected to UV irradiation for 3 min where both, polymerization and cross-linking reaction is carried out. Under UV, dissociation of DMPA (photo initiator) takes place, generating two radicals for each photo initiator molecule. The radicals initiate polymerization of HEMA to form PHEMA and simultaneously poly(ethylene glycol) dimethacrylate (cross linker) is also activated to carry out intermolecular cross-linking of PHEMA chains. After 3 min, hydrogel is delaminated from petridish and dipped into DI water for 1 hr to ensure removal of all the by-products and unreacted reagents.

### Chemical staining of PHEMA hydrogel with 4-[4-(2-Hydroxyethanesulfonyl)-phenylazo]-2,6-dimethoxyphenol

In a typical immobilization procedure, 100 mg of the indicator dye is thoroughly mixed (in a mortar with a pestle) with 1 g concentrated sulfuric acid and left for 30 min at room temperature. This converts the 2-hydroxyethylsulfonyl group of indicator dye into the sulfonate. The mixture is then poured into 900 ml of distilled water and neutralized with 1.6 ml 32% sodium hydroxide solution. Then, 25.0 g of sodium carbonate dissolved in 100 ml water and subsequently, 5.3 ml of 32% sodium hydroxide solution are added. At this stage, PHEMA hydrogel layers are placed into this dyeing solution. Under basic conditions, dye sulfonate is converted into the chemically reactive vinylsulfonyl derivative, and simultaneously, Michael addition of the vinylsulfonyl group with reactive groups of the polymer, (e.g. the hydroxyl groups of the PHEMA hydrogel) takes place. After 12 h, the coloured layers are removed from the dyeing bath and washed several times with distilled water.

At this stage, the dye molecule is chemically linked to the cross-linked polymer matrix. Also due to hydrogel's ability to absorb aqueous solutions, the dye gets physically loaded into the matrix. This is non-covalent type of binding of dye to the polymer as shown herein below. After enough washing, leaching of dye from the hydrogel is stopped, and at this stage coloured hydrogel is cut into small pieces to be used in nucleic acid testing.

FIG. 19 provides a schematic representation of physical entrapment and chemical linkage pH indicator dye to the cross-linked polymer matrix (FIG. 19).

### Example 7

### Test of the Hydrogel in a LAMP Reaction

The reactions are set up to use lambda primer set and lambda DNA. About 10 billion copies lambda DNA are mixed with reaction components with the presence of a slab of hydrogel (tube 2). The dNTPs are replaced by a 2.8 mM mixture of (deoxyadenosine triphosphate, deoxyguanosine triphosphate, deoxycytidine triphosphate) in the negative control sample (tube 1). The reaction is held at 63 degree Celsius for 30 mins, and the colour of reaction is observed. The hydrogel slab is about 2 mm×4 mm×1 mm. At the end of the reaction, it is clear that the hydrogel slab changes from magenta to orange with the presence of all four deoxynucleotides, while the colour remains magenta when the missing deoxythymidine triphosphate prevented LAMP reaction.

| | Final concentration | |
|---|---|---|
| Primers mix solution | | |
| Lambda_FIP | 1.6 | uM |
| Lambda_BIP | 1.6 | uM |
| Lambda_LF | 0.8 | uM |
| Lambda_LB | 0.8 | uM |
| Lambda_F3 | 0.2 | uM |
| Lambda_B3 | 0.2 | uM |
| LAMP buffer | | |
| KCl | 50 | mM |
| MgSO4 | 5 | mM |
| NH₄Cl | 5 | mM |
| BSA | 1 | mg/mL |
| Tween 20 | 0.10% | |
| Betain | 1M | |
| Deoxynucleotides | 2.8 | mM |
| Bst polymerase | 32 | U |
| H2O | Fill to 50 | uL |

The colour difference between the reaction versus no reaction when hydrogel slabs are used is provided in FIG. 20.

| | | Before | After |
|---|---|---|---|
| | | LAMP | LAMP |
| K2 | Lamp | Colour | Colour |
| hydrogel | reaction | value | value |
| Tube 1 | No | 5 | 5 |
| Tube 2 | Yes | 5 | 3 |

Example of pH responsive dye conjugated hydrogel of polyurethane on a cellulose acetate ball of 2 mm diameter.

pH responsive dye on polymer ball

| | |
|---|---|
| pH 7 | pH 8.5 |

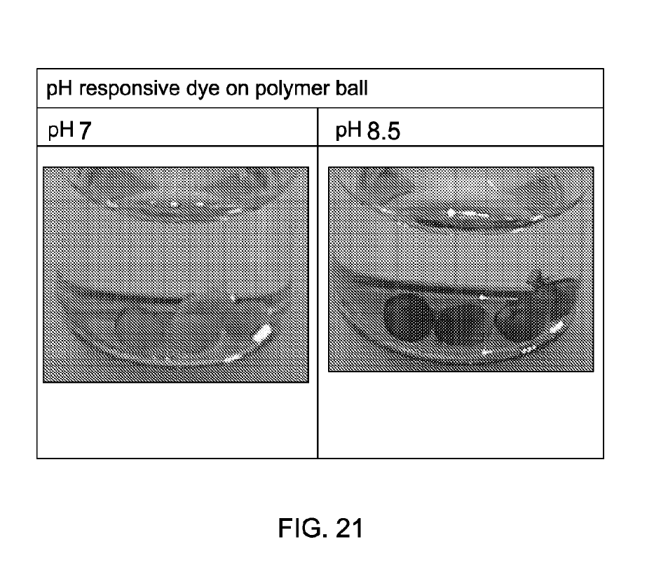

FIG. 21 provides the pH response of the core-shell hydrogel particles. The hydrogel coated cellulose acetate is covalently linked with the pH indicator dye, and the colour of the dye is displayed. At pH 7, the colour is yellow. At pH 8.5, the colour is magenta.

### Lambda Primer Set

Lambda_FIP
5′-CAGCATCCCTTTCGGCATACCAGGTGGCAAGGGTAATGAGG-3′
Lambda_BIP
5′-GGAGGTTGAAGAACTGCGGCAGTCGATGGCGTTCGTACTC-3′
Lambda_F3
5′-GAATGCCCGTTCTGCGAG-3′
Lambda_B3
5′-TTCAGTTCCTGTGCGTCG-3′
Lambda_LF
5′-GGCGGCAGAGTCATAAAGCA-3′
Lambda_LB
5′-GGCAGATCTCCAGCCAGGAACTA-3′

### CYP2C19 Primer Set

| | |
|---|---|
| 2C19_F3 | 5′-CCA GAG CTT GGC ATA TTG TAT C-3′ |
| 2C19_B3 | 5′-AGG GTT GTT GAT GTC CAT-3′ |
| 2C19_FIP.Wild | 5′-CCG GGA AAT AAT CTT TTA ATT TAA |
| | ATT ATT GTT TTC TCT AG-3′ |
| 2C19_BIP.Wild | 5′-CGG GAA CCC GTG TTC TTT TAC TTT |
| | CTC C-3′ |
| 2C19_FIP.Mut | 5′-CTG GGA AAT AAT CTT TTA ATT TAA |
| | ATT ATT GTT TTC TCT AG-3′ |
| 2C19_BIP.Mut | 5′-CAG GAA CCC GTG TTC TTT TAC TTT |
| | CTC C-3′ |
| 2C19_LF | 5′-GAT AGT GGG AAA ATT ATT GC-3′ |
| 2C19_LB | 5′-CAA ATT ACT TAA AAA CCT TGC TT-3′ |

### Primer Sequence:

| | |
|---|---|
| HCV core Forward primer | GTCGCGTAACTTGGGTAAGG |
| HCV core Reverse primer | AAGCTGGGATGGTCAAACAG |

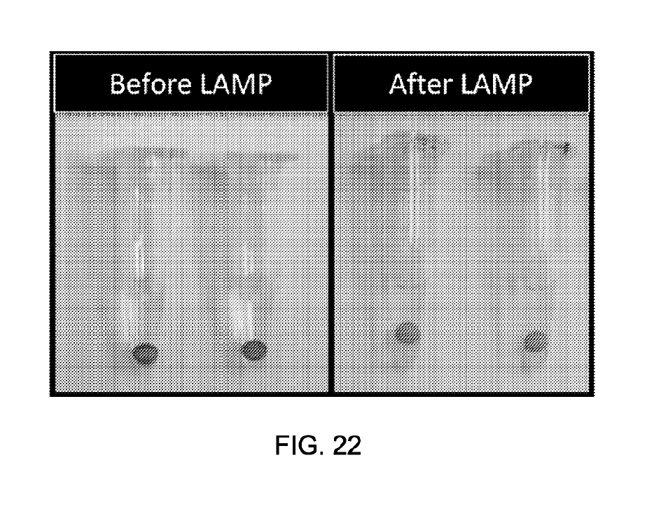

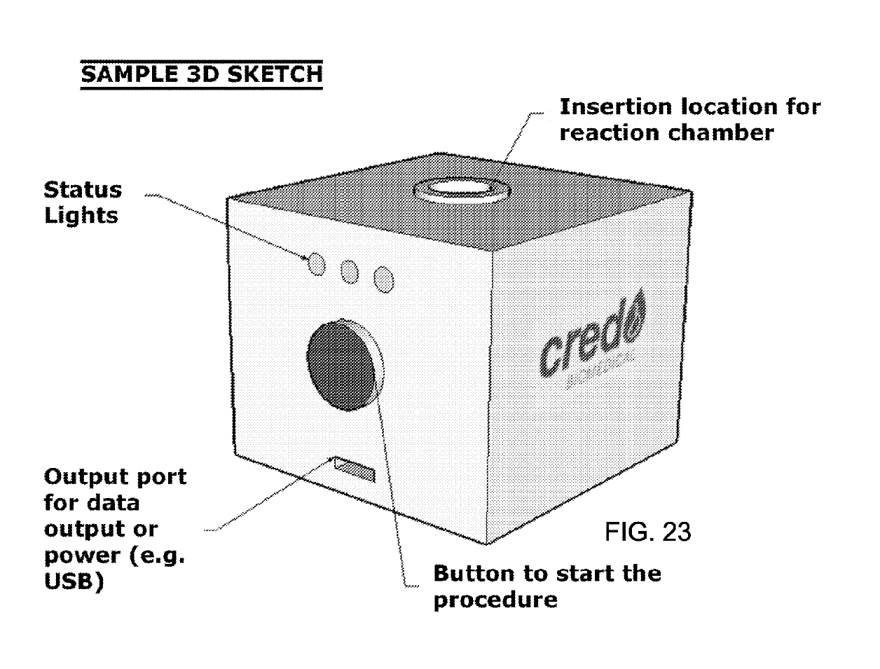

The response of bead in a LAMP reaction is provided in FIG. 22. The bead colour is magenta before the reaction and orange after the reaction.

LAMP-Bead Result

Indicator dye, 4-[4-(2-Hydroxyethanesulfonyl)-phenylazo]-2,6-dimethoxyphenol is immobilized on a cellulose acetate beads. The size of the bead is 2 mm in diameter. The pKa of the dye after immobilization is around 7.5. When the bead is mixed with the 50 micro litre of LAMP reaction mixture (50 mM potassium chloride, 5 mM magnesium sulfate, 5 mM ammonium chloride, 0.1% w/v tween 20, 1M betaine, 2 mM deoxynucleotides, 32 U Bst polymerase, 1 mg/mL bovine serum albumin, 1000 copies of lambda DNA, 1.6 micro M, lambda_FIP primer and lambda_BIP, 0.8 micro M lambda LF and lambda LB primer, 0.2 uM lambda_F3 and lambda_B3 primer, pH 8.5, the colour of the bead is deep magenta. The beads are mixed with all the LAMP reagents and sealed in a micro tube. Two replicates are performed. After the enzyme reaction (63° C. for 45 minutes), the pH change from the enzyme reaction is visually recognizable as bright yellow.

### Example 8

### SNP of MMPI Gene

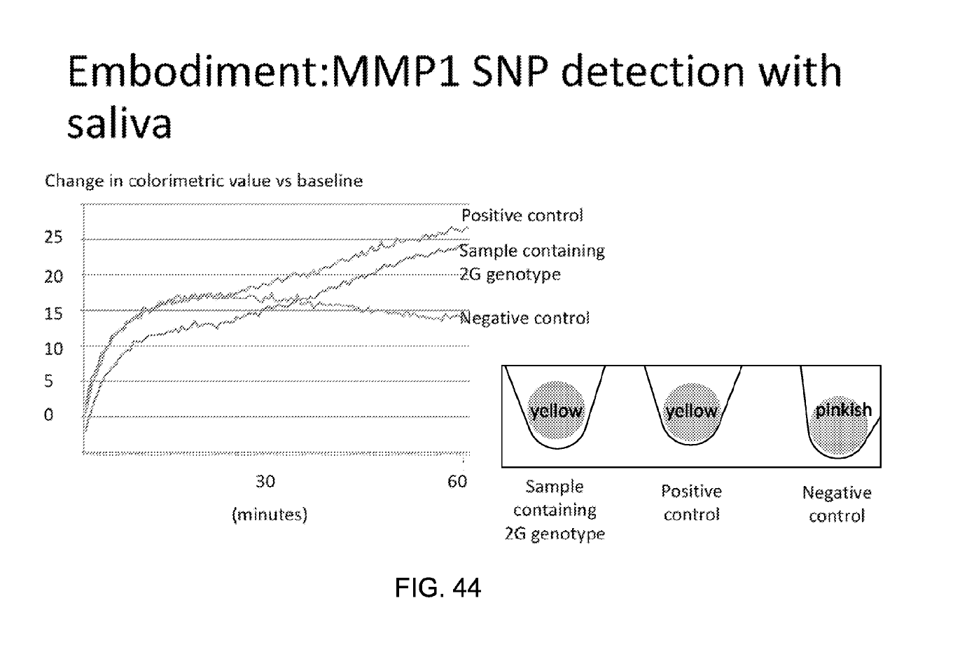

This example involves the detection of an insertion and deletion single nucleotide polymorphism (SNP) on the MMP1 (rs 1799750) gene. The two genotypes of interest are 2G (insertion) and 1G (deletion). The detection method of the present invention is investigated by taking a saliva sample from a human subject. The sample is run against a positive control and negative control, and measurements are taken over a time period of 0 to 60 seconds. (See FIG. 44 for graph results). FIG. 44 shows real time colorimetric readings of 3 containers: one has the tested reaction (sample containing 2G genotype); one is a positive control, and the last is a negative control. This figure shows that the “test” and the “positive control” readings indicate amplification occurs in these containers by the colorimetric changes. The corresponding picture for “test” and “positive control” containers shows that the reactions have proceeded as the colors are yellowish.

On the other hand, the reading does not change very significantly in the “negative control’ container. The corresponding picture shows “negative control” container's remains a pinkish color indicative that the reaction has not proceeded.

Also shown in FIG. 44 is the colorimetric results of the reaction. The first tube is yellow and relates to the 2G reaction. The second tube is yellow and is a positive control, and the third tube is pink indicating no reaction.

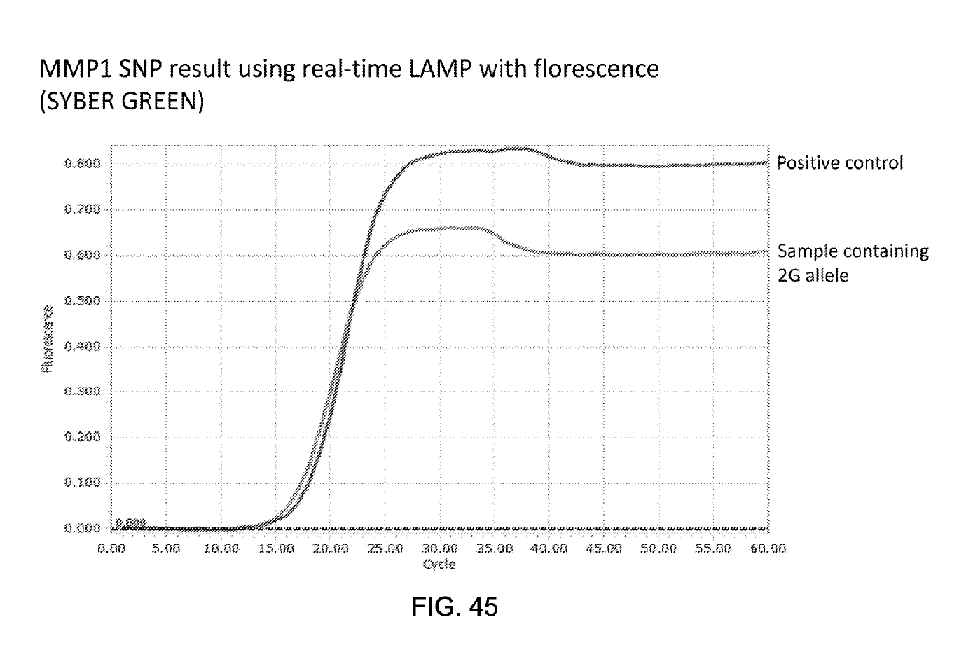

The reaction device of the present invention uses a real-time LAMP fluorescence method. As can be seen in FIG. 45, the 2G reaction is amplified, as indicated by it's parallel observations with a positive control.

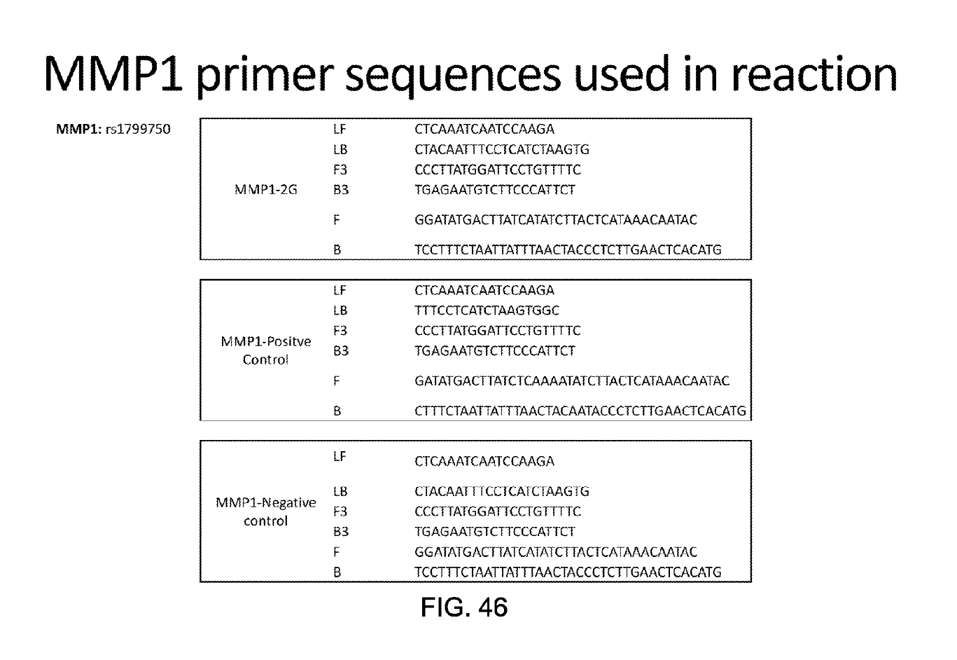

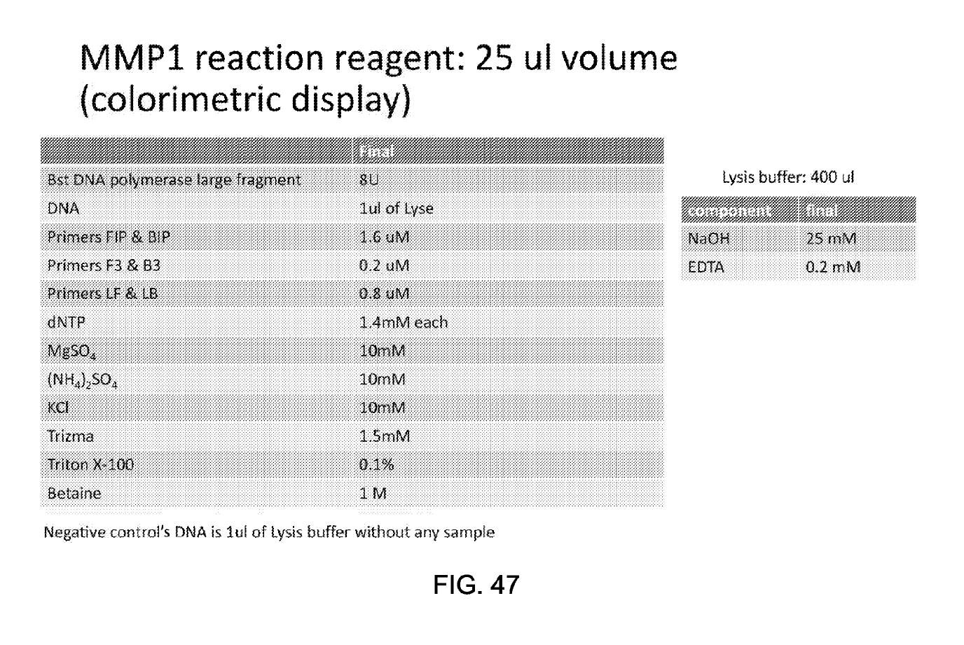

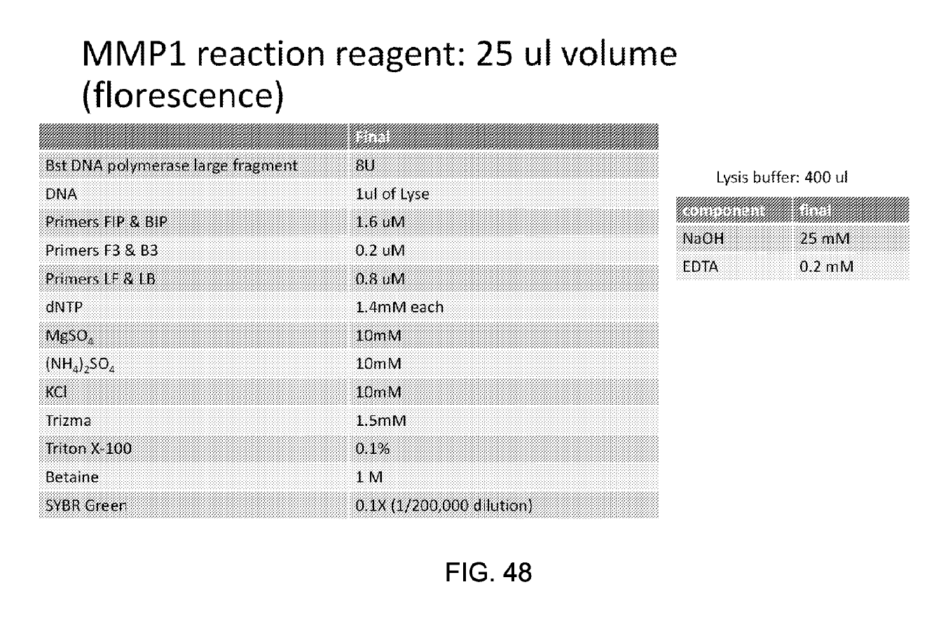

More specifically, in FIG. 46, the primers used for the 2G and controls are provided. FIGS. 47 and 48 provide the list of reagents used in the example as well as the amount of reagent utilized for this example. These data are recorded in real time and through the use of artificial intelligence software with a camera. As an example, FIG. 44 provides an example where the reaction can be stopped at an earlier time frame. Thus, the device provides for monitoring reactions to determine whether the reaction is completed.

### Example 9

### Whole Blood

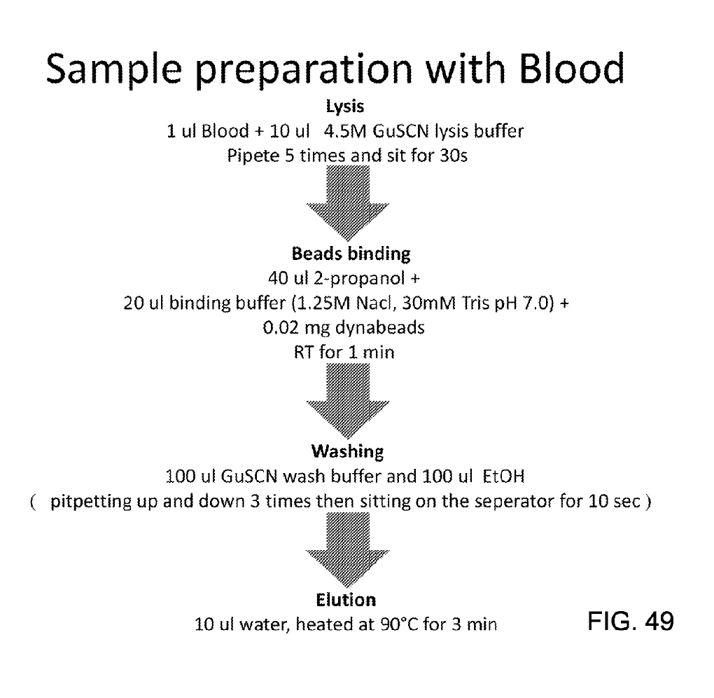

In this example, the device of the present invention utilizes the same sample/reaction container wherein magnetic beads are located (See FIG. 49 for preparations used herein). Blood is lysed in 4.5 M buffer. This sample is pipetted and then rests for 30 s. Thereafter, beads prepared according to FIG. 49 have the blood added to their container. The resultant blood/bead mixture is then washed with buffer and EtOH. The blood/beads mixture is then eluted with water heated to 90° C. for 3 minutes.

It is well known in the art to perform sample preparations for whole blood samples and other samples with magnetic beads. It is much more difficult for other systems to perform sample preparation and DNA amplification in the same container though.

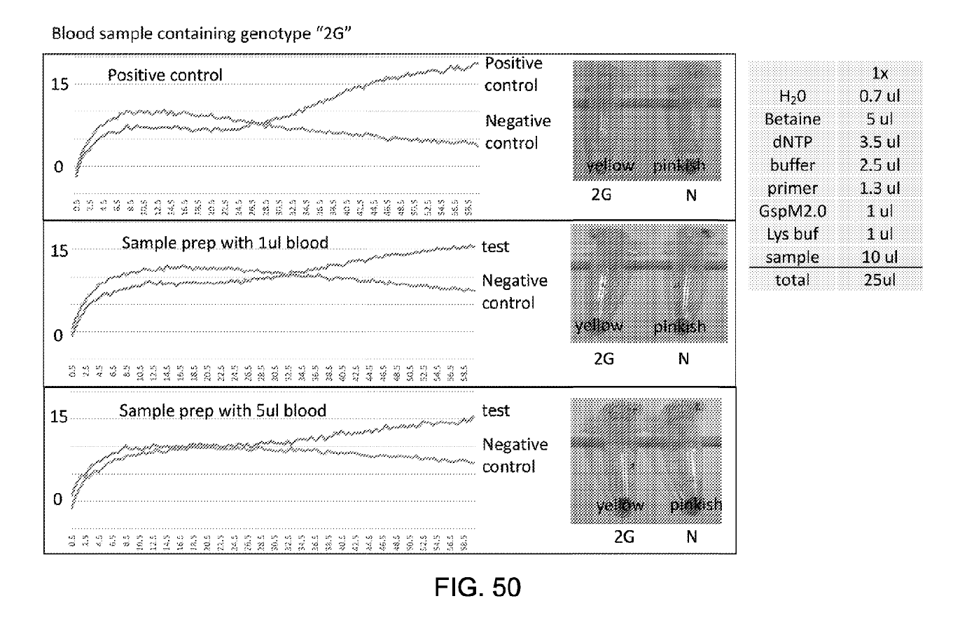

Notice in FIG. 50, that the signal is easily observed with the present invention even when most of the magnetic beads for sample preparation remain in the reaction container during DNA amplification.

Magnet can be used to move the beads around to/from multiple containers if necessary.

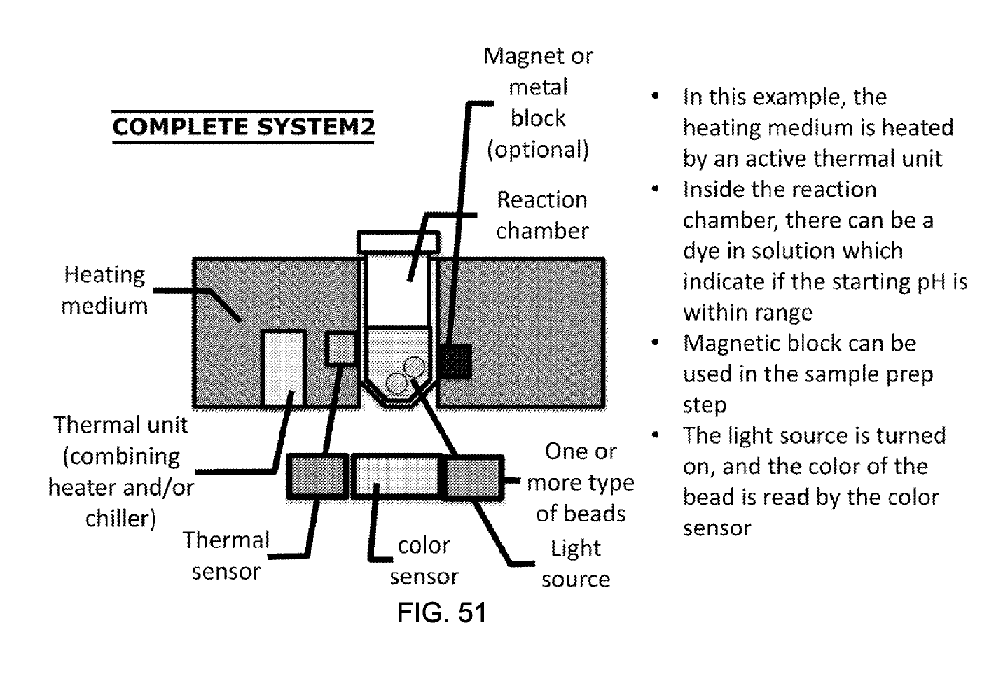

The device found in FIG. 51 is another useful example of the invention described herein.

We claim:

1. A kit to detect biological reactions, said kit comprising: (a) one or more container(s), (b) biological reaction reagents, and (c) at least one pH indicator dye.

2. The kit according to claim 1, wherein the pH indicator dye is potassium 1-hydroxyl-4-[4-(hydroxyethylsulphonyl)-phenylazo]-naphthalene-2-sulphonate or 4-[4-(2-hydroxylethanesulfonyl)-phenylazo]-2,6-dimethoxyphenol or any reactive vinylsulphonyl dye or combination thereof.

3. The kit according to claim 1, wherein said biological reaction measured is an enzymatic reaction.

4. The kit according to claim 1, wherein said biological reaction is nucleic acid amplification.

5. The kit according to claim 1, wherein the reaction is measured by the pH indicator dye in solution, and wherein the dye is immobilized on the surface of films, tube wall or hydrogel immobilized on three dimensional objects, hydrogels, and/or beads.

6. The kit according to claim 1, wherein the pH indicator dye is in solution, lyophilized with said reagents, is pre-mixed in a cocktail solution or is added after the reaction with beads containing said dye on them.

7. A device for or detecting biological reaction, said device comprising: a reaction chamber and at least one thermal unit.

8. The device according to claim 7 wherein the thermal unit is a heater, a chiller or both.

9. The device according to claim 8 additionally comprising at least one opening for reading results.

10. The device according to claim 9 additionally comprising at least one sensor for reading the result.

11. The device according to claim 10, additionally comprising at least one monitor for displaying the result.

12. The device according to claim 11 wherein the opening has at least one light guide.

13. The device according to claim 12 additionally comprising at least one light source and at least one colour sensor as part of the device.

14. The device according to claim 7 wherein said reaction chamber has a pH indicator dye.

15. The device according to claim 14 wherein the device either has said dye located on one or more beads or said dye is not located on the beads.

16. The device according to claim 15, wherein said beads have dye on them; said dye is in solution with magnetic beads; or said magnetic beads have dye on them.

17. The device according to claim 15, wherein said dye has a paramagnetic or ferromagnetic component.

18. The device according to claim 7, additionally comprising a magnet, metal block or magnetic beads.

19. The device according to claim 18 wherein said magnet, metal block or magnetic beads are located in a separate chamber than the reaction chamber.

20. The device of claim 18 wherein said magnet, metal block or magnetic beads are located in the reaction chamber of said device.

21. The device according to claim 18, wherein said magnet or metal block can be moved at various points of the reaction.

22. The device according to claim 18 wherein the thermal unit contains a metal block, liquid, wax or water bath as a thermal medium.

23. The device according to claim 22, wherein the connection between a monitor and the device is wired or wireless.

24. The device according to claim 23, wherein the monitor is a portable device.

25. The device according to claim 24 wherein the portable device is a phone or tablet computer.

26. A device to measure a biological reaction, said device comprising;
a) a light source;
b) a light guide;
c) a colour sensor;
d) a thermal unit for heating, cooling or both;
e) a thermal sensor,
f) one or more reaction chambers containing a reaction solution and beads;
g) an external magnet or metal block or magnetic beads within the reaction chamber;
h) a data port to connect the device to a read ready device; and a
i) heating medium;
wherein said device analyzes measures and records the biological reaction, and wherein said light source and colour sensor are located and guided through the same light guide.

27. The device according to claim 26, additionally comprising a display unit for instructions to use the device and display the results.

28. The device according to claim 27, additionally comprising lights on the device to provide instructions and display the results.

29. The device of claim 28 as a component of a kit to analyze nucleic acid reactions, said kit comprising said device, and containing at least one pH indicator dye.

30. The device of claim 29 as a component of a kit to analyze enzymatic reactions, said kit comprising said device, and containing at least one pH indicator dye.

31. The device according to claim 26 additionally comprising:
a computerized artificial intelligence capability.

32. The device according to claim 31 wherein said computer determines the completion of the reaction measured.

33. The device according to claim 31, wherein said device measures the positive control of the reaction measured, the negative control of the reaction being measured and/or both controls.

34. The device according to claim 31, wherein said device prints and/or illustrates instructions to individuals using said device in order to continue with the reactions based on real time observations.

35. A kit to detect biological reactions, said kit comprising: at least one enzyme friendly pH indicator dye that is enzyme friendly; and a device for carrying out a reaction of the kit.

36. The kit according to claim 35, wherein the enzyme friendly pH indicator dye is potassium 1-hydroxy-4-[4-(hydroxyethylsulphonyl)-phenylazo]-naphthalene-2-sulphonate or 4-[4-(2-hydroxyethanesulfonyl)-phenylazo]-2,6-dimethoxyphenol or any reactive vinylsulphonyl dye or combinations thereof.

37. The kit according to claim 3, additionally comprising: an selected from enzyme friendly pH indicator surface cellulose polymer, hydrogel, metal oxide, polymers or combinations thereof.

38. The kit according to claim 37, wherein the reaction is measured by the detection of pattern changes of the dye.

39. The kit according to claim 38, wherein the enzyme-friendly pH indicator surface is one or more discrete objects in the reaction or in the reaction container.

40. The kit according to claim 39, additionally comprising a hydrogel selected from the group consisting of Poly(2-hydroxyethyl methacrylate) (PHEMA), Polyurethane (PU), Poly(ethylene glycol) (PEG), polyethylene glycol methacrylate (PEGMA), polyethylene glycol dimethacrylate (PEGDMA), polyethylene glycol diacrylate (PEGDA), Poly (vinyl alcohol) (PVA), Poly(vinyl pyrrolidone) (PVP), or Polyimide (PI).

41. The kit according to claim 40, additionally comprising: one or more discrete objects selected from paramagnetic or ferromagnetic components.

42. The kit according to claim 35, further comprising reagents for the biological reaction.

43. The kit according to claim 35, further comprising reagents for biological reaction for nucleotide identification.

44. The kit according to claim 42, wherein the reagent is lyophilized.

45. The kit according to claim 42, wherein the reagent is pre-mixed in a cocktail solution.

46. The kit according to claim 35, wherein the kit is used to detect biological reactions, said kit further comprises (a) one or more container(s), (b) reagents for enzymatic reactions, and (c) at least one pH indicator dye.

47. The kit according to claim 46, wherein the kit additionally has a device for measuring biological reactions of said kit, comprising: (a) a reaction chamber, (b) at least one thermal unit used, and (c) a sensor for measuring changes to the biological reaction.

48. The kit according to claim 47, wherein the reaction is measured by detection of pattern changes of the dye.

49. The kit according to claim 47, the kit further comprising a heater, chiller or both, further comprising a paramagnetic pH indicator dye and an external magnet, and at least one opening for reading the results and at least one sensor for reading the result.

50. The kit according to claim 46, further comprising a paramagnetic pH indicator dye and an external magnet.

51. The kit according to claim 50 wherein the indicator dye is controlled by a magnet outside the container.

52. The kit according to claim 36, wherein the indicator dye is potassium 1-hydroxy-4-[4-(hydroxyethylsulphonyl)-phenylazo]-naphthalene-2-sulphonate or 4-[4-(2-hydroxyethanesulfonyl)-phenylazo]-2,6-dimethoxyphenol or any reactive vinylsulphonyl dye or combinations thereof.

53. The kit according to claim 36 wherein a water bath or wax is additionally added when an isothermal reaction is measured.
